(19) United States
(12) Patent Application Publication
(10) Pub. No.: US 2026/0199358 A1
(43) Pub. Date: Jul. 16, 2026

(54) AP2 ASSOCIATED KINASE 1 INHIBITOR FOR TREATING MUSCULAR DISEASES AND DISORDERS

(71) Applicant: Satellos Bioscience Inc., Toronto (CA)

(72) Inventors: Philip D. LAMBERT, Annapolis, MD (US); Tessa J. MURRAY, Sutton, MA (US)

(21) Appl. No.: 19/431,764

(22) Filed: Dec. 23, 2025

Related U.S. Application Data
(60) Provisional application No. 63/738,454, filed on Dec. 23, 2024.
(60) Provisional application No. 63/772,281, filed on Mar. 14, 2025.
(60) Provisional application No. 63/810,673, filed on May 22, 2025.
(60) Provisional application No. 63/893,499, filed on Oct. 3, 2025.

(51) Int. Cl. A61K 31/519; A61P 21/00

(52) U.S. Cl. CPC A61K 31/519; A61P 21/00

(57) ABSTRACT

The present disclosure provides compositions, doses, and dosing regimens for the use of AAK1 inhibitors in the treatment of muscular diseases and disorders. Also provided are methods of preparation and related kits comprising AAK1 inhibitors.

### CROSS REFERENCE TO RELATED APPLICATIONS

The present application claims the benefit of priority to U.S. Provisional Application No. 63/738,454, filed Dec. 23, 2024, U.S. Provisional Application No. 63/772,281, filed Mar. 14, 2025, U.S. Provisional Application No. 63/810,673, filed May 22, 2025, and U.S. Provisional Application No. 63/893,499, filed Oct. 3, 2025, each of which is incorporated herein by reference in its entirety.

### FIELD

The present disclosure relates to methods of using an AAK1 inhibitor for the treatment of muscular diseases and disorders.

### BACKGROUND

Dystrophin expression within muscle stem cells facilitates their asymmetric division, leading to the production of the progenitor cell population that is ultimately responsible for the repair and regeneration of damaged muscle fibers. A highly druggable protein kinase target called AP2 Associated Kinase 1 (AAK1) was previously identified. Inhibition of AAK1 promoted functional rescue of asymmetric stem cell division, resulting in the robust production of progenitors in vitro and in vivo. There is a need for suitable compositions, formulations, dosing regimens, and methods of use of AAK1 inhibitors to treat patients with muscular diseases or disorders, such as Duchenne muscular dystrophy (DMD).

### SUMMARY

The present disclosure provides compositions and methods for treating disease associated with AAK1.

In one embodiment, the disclosure provides a method of treating Duchenne Muscular Dystrophy (DMD) in a subject in need thereof, comprising administering a therapeutically effective amount of Compound 1,

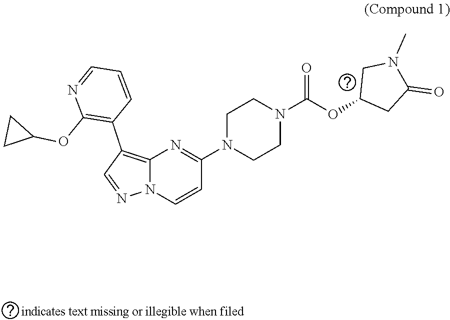 [SMILES: CN1C[C@@H](OC(=O)N2CCN(c3ccn4ncc(-c5cccnc5OC5CC5)c4n3)CC2)CC1=O]

or a pharmaceutically acceptable salt or deuterated form thereof for a treatment period, wherein each treatment period comprises two or more consecutive treatment cycles,
wherein each treatment cycle comprises administering Compound 1 or the pharmaceutically acceptable salt or deuterated form thereof of Compound 1 to the subject for 3, 4, 5, 6, 7, 8, 9, or 10 consecutive days, followed by an interval of 1, 2, 3, or 4 days wherein Compound 1 or the pharmaceutically acceptable salt thereof of Compound 1 is not administered to the subject.

In particular embodiments of any of the methods disclosed herein:
(a) Compound 1 or the pharmaceutically acceptable salt thereof or deuterated form of Compound 1 is administered for at least 3 consecutive days, followed by an interval of at least 1 day wherein Compound 1 or the pharmaceutically acceptable salt thereof or deuterated form of Compound 1 is not administered;
(b) Compound 1 or the pharmaceutically acceptable salt thereof or deuterated form of Compound 1 is administered for at least 4 consecutive days, followed by an interval of at least 1 day wherein Compound 1 or the pharmaceutically acceptable salt thereof or deuterated form of Compound 1 is not administered;
(c) Compound 1 or the pharmaceutically acceptable salt thereof or deuterated form of Compound 1 is administered for at least 5 consecutive days, followed by an interval of at least 1 day wherein Compound 1 or the pharmaceutically acceptable salt thereof or deuterated form of Compound 1 is not administered; or
(d) Compound 1 or the pharmaceutically acceptable salt thereof or deuterated form of Compound 1 is administered for at least 6 consecutive days, followed by an interval of at least 1 day wherein Compound 1 or the pharmaceutically acceptable salt thereof or deuterated form of Compound 1 is not administered.

In particular embodiments, the treatment period comprises at least one 7-day treatment cycle, wherein Compound 1 or the pharmaceutically acceptable salt thereof or deuterated form of Compound 1 is administered for 3 or more days of each 7-day treatment cycle and not administered for 1 or more day of each 7-day treatment cycle.

In certain embodiments, each treatment cycle is a 7-day treatment cycle wherein:
(a) Compound 1 or the pharmaceutically acceptable salt thereof or deuterated form of Compound 1 is administered for 3 consecutive days, followed by an interval of 4 days wherein Compound 1 or the pharmaceutically acceptable salt thereof or deuterated form of Compound 1 is not administered;
(b) Compound 1 or the pharmaceutically acceptable salt thereof or deuterated form of Compound 1 is administered for 4 consecutive days, followed by an interval of 3 days wherein Compound 1 or the pharmaceutically acceptable salt thereof or deuterated form of Compound 1 is not administered;
(c) Compound 1 or the pharmaceutically acceptable salt thereof or deuterated form of Compound 1 is administered for 5 consecutive days, followed by an interval of 2 days wherein Compound 1 or the pharmaceutically acceptable salt thereof or deuterated form of Compound 1 is not administered; or
(d) Compound 1 or the pharmaceutically acceptable salt thereof or deuterated form of Compound 1 is administered for 6 consecutive days, followed by an interval of 1 day wherein Compound 1 or the pharmaceutically acceptable salt thereof or deuterated form of Compound 1 is not administered.

In certain embodiments, for each 7-day treatment cycle, Compound 1 or the pharmaceutically acceptable salt thereof or deuterated form of Compound 1 is administered for 5 consecutive days, followed by an interval of 2 days wherein Compound 1 or the pharmaceutically acceptable salt thereof or deuterated form of Compound 1 is not administered.

In certain embodiments of any of the methods disclosed herein, Compound 1 or the pharmaceutically acceptable salt thereof or deuterated form of Compound 1 is administered once daily. In certain embodiments, 10-400 mg of Compound 1 or the pharmaceutically acceptable salt thereof or deuterated form of Compound 1 is administered to the subject daily. In certain embodiments, about 10 mg, about 50 mg, about 60 mg, about 100 mg, about 120 mg, about 150 mg, about 180 mg, about 200 mg, about 240 mg, about 300 mg, or about 400 mg of Compound 1 or the pharmaceutically acceptable salt thereof or deuterated form of Compound 1 is administered to the subject daily.

In certain embodiments of the disclosed methods, for each treatment cycle, Compound 1 or the pharmaceutically acceptable salt thereof or deuterated form of Compound 1 is orally administered once daily at a dose of about 60 mg for five consecutive days, followed by two days during which the subject is not administered the Compound 1 or the pharmaceutically acceptable salt thereof or deuterated form of Compound 1.

In certain embodiments of the disclosed methods, for each treatment cycle, Compound 1 or the pharmaceutically acceptable salt thereof or deuterated form of Compound 1 is orally administered once daily at a dose of about 120 mg for five consecutive days, followed by two days during which the subject is not administered the Compound 1 or the pharmaceutically acceptable salt thereof or deuterated form of Compound 1.

In certain embodiments of any of the disclosed methods, Compound 1 or the pharmaceutically acceptable salt thereof or deuterated form of Compound 1 is administered orally. In certain embodiments of any of the disclosed methods, Compound 1 is administered orally.

In particular embodiments, each treatment cycle is the same or different.

In certain embodiments of any of the disclosed methods, force generation by muscles is increased, forced vital capacity (FVC) is increased, and/or 99th Percentile Maximum Effort is increased in the subject after administration of Compound 1 or the pharmaceutically acceptable salt thereof or deuterated form of Compound 1. In some embodiments, the force generation by muscles is measured by upper extremity dynamometry. In some embodiments, the FVC is measured by spirometry. In some embodiments, the 99th Percentile Maximum Effort is measured by a wearable monitoring device.

In certain embodiments of any of the disclosed methods, the blood plasma level of Compound 1 or the pharmaceutically acceptable salt thereof or deuterated form of Compound 1 in the subject is:
(a) above the IC50 for a period of time within the 24 hours following each administration of the Compound 1 or the pharmaceutically acceptable salt thereof or deuterated form of Compound 1, and wherein the blood plasma level is below the IC50 during the interval wherein Compound 1 or the pharmaceutically acceptable salt thereof or deuterated form of Compound 1 is not administered; and/or

(b) above about 130 ng/ml for a period of time within the 24 hours following each administration of Compound 1 or the pharmaceutically acceptable salt thereof or deuterated form of Compound 1 is administered, and wherein the blood plasma level is below about 130 ng/ml during the interval wherein Compound 1 or the pharmaceutically acceptable salt thereof or deuterated form of Compound 1 is not administered.

In certain embodiments of any of the disclosed methods, blood plasma level of Compound 1 or the pharmaceutically acceptable salt thereof or deuterated form of Compound 1 is above the IC50 or above about 130 ng/ml for at least about 2 hours, 4 hours, or 6 hours during the 24 hours following each administration of the Compound 1 or the pharmaceutically acceptable salt thereof or deuterated form of Compound 1; and/or the blood plasma level is above the IC50 or above about 130 ng/ml for at least about 6-8 hours during the 24 hours following each administration of the Compound 1 or the pharmaceutically acceptable salt thereof or deuterated form of Compound 1.

In certain embodiments, the subject in need thereof is ambulatory, and in certain embodiments, the subject is non-ambulatory.

In certain embodiments of any of the disclosed methods, the pharmaceutically acceptable salt of Compound 1 is a crystalline or polymorph form. In certain embodiments of any of the disclosed methods, the pharmaceutically acceptable salt of Compound 1 is an oxalate salt. In certain embodiments of any of the disclosed methods, Compound 1 is administered.

In related embodiments, the disclosure provides a kit for treatment of Duchenne muscular dystrophy (DMD), comprising at least 3 compound-containing units and optionally at least 1 compound-free unit, wherein each of said compound-containing units comprises Compound 1, or a pharmaceutically acceptable salt or deuterated form thereof or the crystalline or polymorph form of Compound 1, and each of said compound-free unit does not comprise Compound 1 or the pharmaceutically acceptable salt thereof or deuterated form of Compound 1.

In certain embodiments, the kit comprises:
3 compound-containing units and optionally 4 compound-free units;
4 compound-containing units and optionally 3 compound-free units;
5 compound-containing units and optionally 2 compound-free units; or
6 compound-containing units and optionally 1 compound-free units.

In certain embodiments, the kit comprises 12 to 24 compound-containing units and optionally 4 to 16 compound-free units.

In certain embodiments, the kit comprises:
12 compound-containing units and optionally 16 compound-free units;
16 compound-containing units and optionally 12 compound-free units;
20 compound-containing units and optionally 8 compound-free units; or
24 compound-containing units and optionally 4 compound-free units.

In certain embodiments, the kit comprises 20 compound-containing units and optionally 8 compound-free units, wherein each compound-containing unit comprises about 60 mg or about 120 mg of Compound 1 or the pharmaceutically acceptable salt thereof or deuterated form of Compound 1.

In particular embodiments of any of the disclosed kits, each compound-containing unit comprises 10-400 mg of Compound 1 or the pharmaceutically acceptable salt thereof or deuterated form of Compound 1. In certain embodiments, each of the compound-containing units comprises about 10 mg, about 50 mg, about 60 mg, about 120 mg, about 150 mg, about 180 mg, about 240 mg, about 300 mg, or about 400 mg of Compound 1 or the pharmaceutically acceptable salt thereof or deuterated form of Compound 1. In particular embodiments, the compound-containing units and compound-free units of the kit are designed to be taken orally once per day. In certain embodiments, each of the compound-containing units comprises the same amount of Compound 1 or the pharmaceutically acceptable salt thereof or deuterated form of Compound 1.

In certain embodiments of any of the disclosed kits, the pharmaceutically acceptable salt of Compound 1 is a crystalline or polymorph form. In certain embodiments of any of the disclosed kits, the pharmaceutically acceptable salt of Compound 1 is an oxalate salt.

In another related embodiment, the disclosure provides a method of increasing the force generation by the skeletal muscles including diaphragm of a subject having Duchenne Muscular Dystrophy (DMD), comprising administering a therapeutically effective amount of Compound 1, or a pharmaceutically acceptable salt thereof or deuterated form of Compound 1, for a treatment period, wherein each treatment period comprises two or more consecutive treatment cycles, wherein each treatment cycle comprises administering Compound 1 or the pharmaceutically acceptable salt thereof to the subject for 3, 4, 5, 6, 7, 8, 9, or 10 consecutive days, followed by an interval of 1, 2, 3, or 4 days wherein Compound 1 or the pharmaceutically acceptable salt thereof or deuterated form thereof is not administered to the subject.

In certain embodiments of any of the disclosed methods, the pharmaceutically acceptable salt of Compound 1 is a crystalline or polymorph form. In certain embodiments of any of the disclosed methods, the pharmaceutically acceptable salt of Compound 1 is an oxalate salt. In certain embodiments of any of the disclosed methods, Compound 1 is administered.

### BRIEF DESCRIPTION OF THE DRAWINGS

FIG. 1A provides a graph showing the number of MyoG+ cells per fiber after treatment with Compound 2 or Compound 1. FIG. 1B provides a graph showing the number of Pax7+ cells after treatment with Compound 2 or Compound 1. FIG. 1C provides a graph showing the % of MyoG cells per fiber after treatment with Compound 2 or Compound 1.

FIG. 2A provides a graph showing the plasma concentrations of Compound 2 in mice administered various doses of Compound 2 and a table of calculated pharmacokinetic (PK) parameters. FIG. 2B provides a graph showing the plasma concentrations of Compound 1 in mice administered various doses of Compound 1 and a table of calculated pharmacokinetic (PK) parameters.

FIG. 3A provides a graph showing the plasma concentrations of Compound 2 in rats administered various doses of Compound 2 and a table of calculated pharmacokinetic (PK) parameters. FIG. 3B provides a graph showing the plasma concentrations of Compound 1 in rats administered various doses of Compound 1 and a table of calculated pharmacokinetic (PK) parameters.

FIG. 4A provides a graph showing the plasma concentrations of Compound 2 in beagles administered various doses of Compound 2 and a table of calculated pharmacokinetic (PK) parameters. FIG. 4B provides a graph showing the plasma concentrations of Compound 1 in beagles administered various doses of Compound 1 and a table of calculated pharmacokinetic (PK) parameters.

FIG. 5A provides a graph showing maximal force production in mice after treatment with various doses of Compound 1 (Cmpd. 1). FIG. 5B provides a graph showing force production at 150 Hz in mice after treatment with various doses of Compound 1.

FIG. 6A provides a graph showing the average body weights of each experimental group of mdx mice at the start of a dosing regimen study. FIG. 6B provides a graph showing the average body weights each experimental group of mdx mice at the midpoint (after 4 weeks of treatment) of a dosing regimen study. FIG. 6C provides a graph showing the average body weights each experimental group of mdx mice at the endpoint (after 8 weeks of treatment) of a dosing regimen study.

FIG. 7 provides a graph of daily body weights of mdx across all experimental groups throughout the length of a dosing regimen study.

FIG. 8A provides a graph showing maximal force production in mdx mice after 4 weeks of treatment with Compound 1 at various dosing intervals. FIG. 8B provides a graph showing maximal force production normalized to body weight in mdx mice after 4 weeks of treatment with Compound 1 at various dosing intervals. FIG. 8C provides a graph showing maximal force production normalized to leg length in mdx mice after 4 weeks of treatment with Compound 1 at various dosing intervals.

FIG. 9A provides a graph showing maximal force production in mdx mice after 6 weeks of treatment with Compound 1 at various dosing intervals. FIG. 9B provides a graph showing maximal force production normalized to body weight in mdx mice after 6 weeks of treatment with Compound 1 at various dosing intervals. FIG. 9C provides a graph provides a graph showing maximal force production normalized to leg length in mdx mice after 6 weeks of treatment with Compound 1 at various dosing intervals.

FIG. 10A provides a graph showing maximal force production in mdx mice after 8 weeks of treatment with Compound 1 at various dosing intervals. FIG. 10B provides a graph showing maximal force production normalized to body weight in mdx mice after 8 weeks of treatment with Compound 1 at various dosing intervals. FIG. 10C provides a graph showing maximal force production normalized to leg length in mdx mice after 8 weeks of treatment with Compound 1 at various dosing intervals. FIG. 10D provides a graph showing maximal force production normalized to muscle weight in mdx mice after 8 weeks of treatment with Compound 1 at various dosing intervals.

FIG. 11A provides a graph showing the weight of the gastrocnemius muscles in mdx mice treated for 8 weeks with Compound 1 at various dosing intervals. FIG. 11B provides a graph showing the weight of the tibialis anterior muscles in mdx mice treated for 8 weeks with Compound 1 at various dosing intervals.

FIG. 12 provides a graph comparing blood plasma levels of Compound 1 in mice after treatment with an efficacious dose and in healthy volunteers after Single Ascending Dose (SAD) dosing. At each time point with two circles, the lower circle is healthy volunteers and the upper circle is mice.

FIG. 13A provides a graph showing mean plasma concentration at Day 1 and Day 7 in healthy volunteers treated with 60 mg of Compound 1 in Part B of the study. FIG. 13B provides a graph showing mean plasma concentration at Day 1 and Day 7 in healthy volunteers treated with 120 mg of Compound 1 in Part B of the study. FIG. 13C provides a graph showing mean plasma concentration at Day 1 and Day 7 in healthy volunteers treated with 180 mg of Compound 1 in Part B of the study. In each graph, the top line is Day 7, and the bottom line is Day 1.

FIG. 14 provides a boxplot from a simulation study showing predicted mean time over IC50 for healthy volunteers and DMD patients of different age groups administered one of four doses of Compound 1.

### DETAILED DESCRIPTION

### Overview

Compound 1 is an AP2 Associated Kinase 1 (AAK1) inhibitor having the structure:

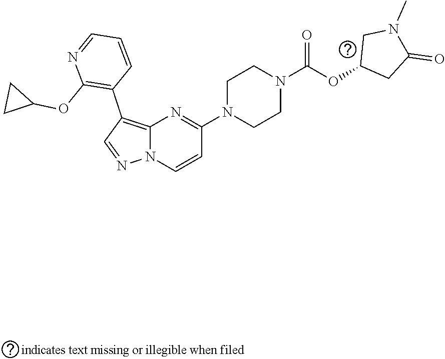 [SMILES: CN1C[C@@H](OC(=O)N2CCN(c3ccn4ncc(-c5cccnc5OC5CC5)c4n3)CC2)CC1=O]

The synthesis and uses of Compound 1 is described in International Application No. PCT/US2024/045579, which is incorporated by reference in its entirety for all purposes.

The present disclosure provides compositions, doses, and dosing regimens for the use of Compound 1, or pharmaceutically acceptable salts or deuterated forms thereof. These doses and dosing regimens are also applicable to the use of crystalline forms of Compound 1, including polymorph forms of Compound 1, in treatment of muscular diseases and disorders. All references to Compound 1 herein also cover all crystalline forms thereof, including cocrystals and polymorphs, such as those described herein. Methods of preparation and related kits comprising Compound 1, or pharmaceutically acceptable salts or deuterated forms thereof, crystalline forms of Compound 1, including cocrystals and polymorph forms of Compound 1 are also provided herein.

### Definitions

The terms “a” and “an” and “the” and similar references used in the context of describing a particular embodiment (especially in the context of certain of the following claims) can be construed to cover both the singular and the plural, unless specifically noted otherwise.

The term “or” as used herein, including the claims, is used to mean “and/or” unless explicitly indicated to refer to alternatives only or the alternatives are mutually exclusive.

The terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In preferred embodiment, “about” refers to =10% of the recited value (e.g., a recited dose).

Numerical ranges are provided for certain quantities in the present application. It is to be understood that these ranges comprise all subranges therein. Thus, the range “10 to 400,” also referred to as “10-400,” includes all possible ranges therein (e.g., 11-399, 12-398, 13-397, 14-396, 15-395, 20-300, 40-200, etc.). Furthermore, all values within a given range may be an endpoint for the range encompassed thereby (e.g., the range 10-400 includes the ranges with endpoints such as 10-300, 50-400, etc.). All ranges provided herein also include all discreet numerical values that fall within the given range, including the endpoints (e.g., the range 10-400 includes 10, 50, 60, 120, 150, 180, 240, 300, 400, etc.).

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

As used herein, the term “cocrystal” refers to a crystalline solid comprising Compound 1 and a coformer in the same crystal lattice. The coformer of the present disclosure is generally a pharmaceutically acceptable Brønsted acid including, but not limited to, oxalic acid. A “Compound 1 cocrystal” as used herein is distinct from a “Compound 1 salt,” which comprises charged-balanced charged species. The species making up a cocrystal typically are neutral, and are generally held together by weak, reversible, non-covalent interactions. The weak interaction generally includes, e.g., hydrogen bonding, van der Waals forces, π-π interactions, and/or halogen bond interactions. Cocrystals can therefore be distinguished from salt, that an ionic bond framework, by the absence of a proton transfer between the chemical species (i.e., Compound 1 and the coformer). In some embodiments, the pharmaceutically acceptable organic acid that is the coformer is an organic acid commonly used to make pharmaceutically acceptable salts, e.g., those described herein.

The terms “administer” or “administration” refer to the act of injecting or otherwise physically delivering a substance as it exists outside the body (e.g., a formulation of the invention) into a patient, such as by oral, mucosal, intradermal, intravenous, subcutaneous, intramuscular delivery and/or any other method of physical delivery described herein or known in the art.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition. In embodiments, “treating” means achieving a clinically relevant reduction in at least one symptom of a condition in a subject.

An “effective amount” means the amount of a compound or formulation according to the disclosure that, when administered to a patient for treating a state, disorder or condition is sufficient to effect such treatment. The term “therapeutically effective” applied to dose or amount refers to that quantity of a compound or pharmaceutical formulation that is sufficient to result in a desired clinical benefit after administration to a patient in need thereof.

The terms “subject” and “patient” are used interchangeably. As used herein, a subject is preferably a mammal, such as a non-primate (e.g., cows, pigs, horses, cats, dogs, rats, etc.) or a primate (e.g., monkey and human), most preferably a human. In one embodiment, the subject is a mammal, preferably a human, having DMD.

As used herein, a “subject” can be a human, non-human primate, mammal, rat, mouse, cow, horse, pig, sheep, goat, dog, cat and the like. “Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

Doses of Compound 1 or Compound 2 described herein refer to the weight of the free base form of Compound 1 or Compound 2. In embodiments where a pharmaceutically acceptable salt of Compound 1 or Compound 2 is administered, it is understood that the pharmaceutically acceptable salt is administered in an amount that provides an equivalent dose of the free base form of Compound 1 or Compound 2. The claims and disclosure encompass such doses of pharmaceutically acceptable salts. For example, if a claim recites administering 50 mg of Compound 1, or a pharmaceutically acceptable salt thereof, the claim encompasses the dose of the pharmaceutically acceptable salt that provides 50 mg of Compound 1 in the free base form.

### Methods

Provided herein, inter alia, are dosing regimens for treatment with AAK1 inhibitors, wherein a subject is treated for a period of time, followed by a period of time wherein said AAK1 inhibitors are not administered to the subject, before resuming administration of the AAK1 inhibitors. For treating diseases such as muscular dystrophies with AAK1 inhibitors, such as Compound 1 or Compound 2 and pharmaceutically acceptable salts and deuterated forms thereof, the inventors have determined that such cyclical regimens, with intervening periods of non-treatment where the active drug compound is not administered, are advantageous. The methods disclosed herein comprise administering Compound 1 or Compound 2 or a pharmaceutically acceptable salt or deuterated form thereof to a subject. The methods may also be practiced by administering crystalline forms of Compound 1, including polymorphic forms of Compound 1, to a subject. Compound 1 or a pharmaceutically acceptable salt or deuterated form thereof, crystalline forms of Compound 1, including cocrystal and polymorphic forms of Compound 1 inhibit an activity of AAK1. In addition, any of the methods disclosed herein may be practiced using any other AAK1 inhibitor, including but not limited to Compound 2 or a pharmaceutically acceptable salt or deuterated form thereof.

The methods may be used to treat or manage a disease or a disorder, e.g., a disease or disorder mediated by AAK1 activity, comprising administering to a subject in need thereof, Compound 1 or Compound 2 or a pharmaceutically acceptable salt or deuterated form thereof, or a crystalline or polymorph form of Compound 1.

In some embodiments, the disease or a disorder mediated by AAK1 activity is a muscular dystrophy. Muscular dystrophies are genetic diseases characterized by progressive weakness and degeneration of the skeletal or voluntary muscles which control movement. The muscles of the heart and some other involuntary muscles are also affected in some forms of muscular dystrophy. In many cases, the histological picture shows variation in fiber size, muscle cell necrosis and regeneration, and often proliferation of connective and adipose tissue. In some embodiments, the muscular dystrophy is Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSHD), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy. In some embodiments, the muscular dystrophy is DMD.

In some embodiments, the disease or disorder mediated by AAK1 activity is a neurodegenerative disease. In some embodiments, the disease or disorder mediated by AAK1 activity is diabetic neuropathy.

The present disclosure also provides method for increasing skeletal muscle tissue growth or regeneration in a subject, comprising administering to the subject Compound 1 or a pharmaceutically acceptable salt or deuterated form thereof.

In some embodiments, the subject has a muscular dystrophy. In some embodiments, the muscular dystrophy is Duchenne muscular dystrophy (DMD), Becker muscular dystrophy (BMD), Emery-Dreifuss muscular Landouzy-Dejerine dystrophy, muscular dystrophy, facioscapulohumeral muscular dystrophy (FSHD), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy. In some embodiments, the subject has DMD.

In specific embodiments, the present disclosure provides methods for treating or managing a disease or disorder mediated by AAK1 activity, e.g., a neurodegenerative disease or muscular dystrophy, comprising administering to a subject in need thereof an effective amount of Compound 1 or Compound 2 or a pharmaceutically acceptable salt or deuterated form thereof, including a crystalline form thereof (e.g., co-crystal or polymorph forms). The Compound 1 or pharmaceutically acceptable salt or deuterated form thereof, including a crystalline form thereof (e.g., co-crystal and polymorph forms) may be administered by any of the routes and in any of the dosages and dosing regimens disclosed herein. In particular embodiments, it is administered orally.

The methods provide for administering Compound 1 or a pharmaceutically acceptable salt or deuterated form thereof, including a crystalline form thereof (e.g., co-crystal and polymorph forms) for at least one treatment period. In embodiments, a treatment period comprises two or more consecutive treatment cycles, wherein each treatment cycle comprises administering Compound 1 or the pharmaceutically acceptable salt or deuterated form thereof, including a crystalline form thereof (e.g., co-crystal and polymorph forms), to the subject for 3, 4, 5, 6, 7, 8, 9, or 10 consecutive days, followed by an interval of 1, 2, 3, or 4 days wherein Compound 1 or the pharmaceutically acceptable salt or deuterated form thereof is not administered to the subject. In particular embodiments, a treatment period comprises two or more treatment cycles, and during each treatment cycle, the subject is administered Compound 1 or a pharmaceutically acceptable salt or deuterated form thereof, including a crystalline form thereof (e.g., co-crystal and polymorph forms) for at least 4 consecutive days, e.g., 4 to 6 days, followed by an interval of at least 1 day, e.g., 1 to 3 days of not being administered Compound 1 or a pharmaceutically acceptable salt or deuterated form thereof, including a crystalline form of Compound 1 (e.g., co-crystal and polymorph forms). In particular embodiments, during each treatment cycle, the subject is administered Compound 1 or a pharmaceutically acceptable salt or deuterated form thereof or a crystalline form thereof (e.g., co-crystal and polymorph forms) for 5 consecutive days followed by an interval of 5 days of not being administered Compound 1 or a pharmaceutically acceptable salt or deuterated form thereof, or a crystalline form thereof. In embodiments, the disclosure includes administering Compound 2, or a pharmaceutically acceptable salt or deuterated form thereof, according to the above methods.

In particular embodiments, Compound 1 or a pharmaceutically acceptable salt or deuterated form thereof, or a crystalline form of Compound 1 is administered at a dose of about 20 mg to about 500 mg or about 50 mg to about 400 mg per day on days of administration. In particular embodiments, Compound 1 or a pharmaceutically acceptable salt or deuterated form thereof, or a crystalline form thereof of Compound 1 is administered at a dose of about 50 mg to about 250 mg per day on days of administration. In particular embodiments, Compound 1 or a pharmaceutically acceptable salt or deuterated form thereof, or a crystalline form thereof of Compound 1 is administered at a dose of about 60 mg to about 240 mg per day on days of administration. In particular embodiments, Compound 1 or a pharmaceutically acceptable salt or deuterated form thereof, or a crystalline form thereof of Compound 1 is administered at a dose of about 100 mg to about 250 mg per day on days of administration. In particular embodiments, Compound 1 or a pharmaceutically acceptable salt or deuterated form thereof, or a crystalline form thereof of Compound 1 is administered at a dose of about 20 mg to about 200 mg per day on days of administration. In particular embodiments, Compound 1 or a pharmaceutically acceptable salt or deuterated form thereof, or a crystalline form thereof of Compound 1 is administered at a dose of about 60 mg per day on days of administration. In particular embodiments, Compound 1 or a pharmaceutically acceptable salt or deuterated form thereof, or a crystalline form thereof of Compound 1 is administered at a dose of about 120 mg per day on days of administration. In embodiments, the disclosure includes administering Compound 2, or a pharmaceutically acceptable salt or deuterated form thereof, according to the above methods.

In particular embodiments, Compound 1 or a pharmaceutically acceptable salt or deuterated form thereof or a crystalline form thereof is administered at a dose sufficient to achieve a blood plasma level greater than the compound's IC50 for a period of time within the next 24 hours, e.g., for at least about 2, 4, or 6 hours, between 2 and 12 consecutive hours, between 6 and 12 consecutive hours, or between 6 and 8 consecutive hours, e.g., about 6, about 7, about 8, about 9, about 10, about 11, or about 12 consecutive hours, but for less than 24 hours, e.g., between 16 and 18 consecutive hours, less than 12 consecutive hours, less than 16 consecutive hours, or less than 18 consecutive hours, following each administration. In particular embodiments, the IC50 is about 130 ng/mL. In particular embodiments, the compound is Compound 1, and it's IC50 is about 130 ng/mL. In embodiments, the disclosure includes administering Compound 2, or a pharmaceutically acceptable salt or deuterated form thereof, according to the above methods.

In particular embodiments, Compound 1 or a pharmaceutically acceptable salt or deuterated form thereof or a crystalline form thereof is administered at a dose sufficient to achieve a therapeutically effective blood plasma level for a period of time within the next 24 hours, e.g., at least about 2, 4, or 6 hours, between 2 and 12 consecutive hours, between 6 and 12 consecutive hours, or between 6 and 8 consecutive hours, e.g., about 6, about 7, about 8, about 9, about 10, about 11, or about 12 consecutive hours, but for less than 24 hours, e.g., between 16 and 18 consecutive hours, less than 12 consecutive hours, less than 16 consecutive hours, or less than 18 consecutive hours, following each administration. In embodiments, the disclosure includes administering Compound 2, or a pharmaceutically acceptable salt or deuterated form thereof, according to the above methods.

In particular embodiments, Compound 1 or a pharmaceutically acceptable salt or deuterated form thereof, or a crystalline thereof is administered at a dose sufficient to achieve a blood plasma level of about 100 ng/ml to about 200 ng/ml, about 100 ng/ml to about 500 ng/ml, about 130 ng/ml to about 500 ng/mL, or about 130 ng/mL to about 200 ng/ml, for a period of time within the next 24 hours, e.g., at least about 2, 4, or 6 hours, between about 2 and about 20 consecutive hours, between about 6 and about 20 consecutive hours, between about 6 and about 12 consecutive hours, between about 6 and about 16 consecutive hours, or between 6 and 8 consecutive hours, e.g., about 6, about 7, about 8, about 9, about 10, about 11, or about 12 consecutive hours, but for less than 24 hours, e.g., between 16 and 18 consecutive hours, less than 12 consecutive hours, less than 16 consecutive hours, or less than 18 consecutive hours, following each administration. In particular embodiments, a blood plasma level of greater than or equal to 130 ng/ml is achieved for about 6-8 hours or about 6-12 hours, and a blood plasma level of less than 130 ng/ml is maintained for the remaining hours of a 24 hour time period following administration. In embodiments, the disclosure includes administering Compound 2, or a pharmaceutically acceptable salt or deuterated form thereof, according to the above methods.

In some embodiments, the muscular dystrophy is selected from the group consisting of: Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSHD), Limb-Girdle muscular dystrophies, von Graefe-Fuchs muscular dystrophy, oculopharyngeal muscular dystrophy (OPMD), Myotonic dystrophy (Steinert's disease) and a congenital muscular dystrophy. In specific embodiments, the muscular dystrophy is DMD.

In some embodiments, the skeletal muscle tissue of the subject is damaged or injured as a result of physical injury or accident, disease, gene mutation, infection, over-use, loss of blood circulation, muscle atrophy, muscle wasting, dystrophic muscle, or ageing.

In some embodiments, the methods provided herein comprising administering Compound 1 or a pharmaceutically acceptable salt or deuterated form thereof, or a crystalline or polymorph form of Compound 1 to a subject increase asymmetric cell division of skeletal muscle stem cells of the subject after administration. In some embodiments, the stem cells are muscle stem cells, retinal stem cells, neural stem cells, hematopoietic stem cells, intestinal stem cells, epidermal stem cells, or cancer or tumor stem cells. In certain embodiments, the stem cells are muscle stem cells or satellite cells. In some embodiments of the present methods, Compound 1 or pharmaceutically acceptable salt or deuterated form thereof, or crystalline or polymorph form of Compound 1 does not substantially inhibit proliferation or cell cycle progression of the subject's skeletal muscle stem cells.

In some embodiments, the methods provided herein comprising administering Compound 2 or a pharmaceutically acceptable salt or deuterated form thereof, or a crystalline or polymorph form of Compound 2 to a subject increase asymmetric cell division of skeletal muscle stem cells of the subject after administration. In some embodiments, the stem cells are muscle stem cells, retinal stem cells, neural stem cells, hematopoietic stem cells, intestinal stem cells, epidermal stem cells, or cancer or tumor stem cells. In certain embodiments, the stem cells are muscle stem cells or satellite cells. In some embodiments of the present methods, Compound 2 or pharmaceutically acceptable salt or deuterated form thereof, or crystalline or polymorph form of Compound 2 does not substantially inhibit proliferation or cell cycle progression of the subject's skeletal muscle stem cells.

In some embodiments, skeletal muscle stem cells within the skeletal muscle tissue of the subject have reduced asymmetric cell division as compared to normal, healthy skeletal muscle stem cells. In certain embodiments, the skeletal muscle stem cells are damaged or injured skeletal muscle stem cells or are present within damaged or injured skeletal muscle tissue. In some embodiments, the muscle tissue is damaged or injured as a result of: physical injury or accident, disease, gene mutation, infection, over-use, loss of blood circulation, muscle atrophy, muscle wasting, dystrophic muscle, or ageing. In some embodiments, the skeletal muscle stem cells are diseased skeletal muscle stem cells comprising a mutation associated with a muscular dystrophy, optionally Duchenne muscular dystrophy (DMD), Becker muscular dystrophy (BMD), Emery-Dreifuss muscular dystrophy, Landouzy-Dejerine muscular dystrophy, facioscapulohumeral muscular dystrophy (FSHD), Limb-Girdle muscular dystrophies, von Graefe-Fuchs muscular dystrophy, oculopharyngeal muscular dystrophy (OPMD), Myotonic dystrophy (Steinert's disease) or a congenital muscular dystrophy.

Accordingly, in one embodiment, the disclosure provides a method for increasing asymmetric cell division of skeletal muscle stem cells of a subject, comprising administering to a subject in need thereof an effective amount of Compound 1 or a pharmaceutically acceptable salt, or deuterated form thereof, including a crystalline form thereof. The Compound 1 or pharmaceutically acceptable salt or deuterated form thereof, including crystalline and polymorph forms thereof, may be administered by any of the routes and in any of the dosages and dosing regimens disclosed herein. In particular embodiments, a treatment period comprises two or more consecutive treatment cycles, wherein each treatment cycle comprises administering Compound 1 or the pharmaceutically acceptable salt or deuterated form thereof to the subject for 3, 4, 5, 6, 7, 8, 9, or 10 consecutive days, followed by an interval of 1, 2, 3, or 4 days wherein Compound 1 or the pharmaceutically acceptable salt or deuterated form thereof is not administered to the subject. In particular embodiments, a treatment period comprises two or more treatment cycles, and during each treatment cycle, the subject is administered Compound 1 or a pharmaceutically acceptable salt, or deuterated form thereof, for at least four consecutive days, e.g., four to six days, followed by an interval of at least one day, e.g., one to three days of not being administered Compound 1 or a pharmaceutically acceptable salt or deuterated form thereof. In particular embodiments, during each treatment cycle, the subject is administered Compound 1 or a pharmaceutically acceptable salt or deuterated form thereof, for five consecutive days followed by an interval of two days of not being administered Compound 1 or a pharmaceutically acceptable salt or deuterated form thereof. In particular embodiments, Compound 1 or a pharmaceutically acceptable salt or deuterated form thereof, is administered orally. In particular embodiments, Compound 1 or a pharmaceutically acceptable salt or deuterated form thereof, or a crystalline form thereof of Compound 1 is administered at a dose of about 20 mg to about 500 mg or about 50 mg to about 400 mg per day on days of administration. In particular embodiments, Compound 1 or a pharmaceutically acceptable salt or deuterated form thereof, or a crystalline form thereof of Compound 1 is administered at a dose of about 50 mg to about 250 mg per day on days of administration. In particular embodiments, Compound 1 or a pharmaceutically acceptable salt or deuterated form thereof, or a crystalline form thereof of Compound 1 is administered at a dose of about 60 mg to about 240 mg per day on days of administration. In particular embodiments, Compound 1 or a pharmaceutically acceptable salt or deuterated form thereof, or a crystalline form thereof of Compound 1 is administered at a dose of about 100 mg to about 250 mg per day on days of administration. In particular embodiments, Compound 1 or a pharmaceutically acceptable salt or deuterated form thereof, or a crystalline form thereof of Compound 1 is administered at a dose of about 20 mg to about 200 mg per day on days of administration. In particular embodiments, Compound 1 or a pharmaceutically acceptable salt or deuterated form thereof is administered at a dose sufficient to achieve a blood plasma level of greater than about 130 ng/ml for at least two, at least four, at least six, or at least eight consecutive hours, e.g., between about two hours and about twelve hours, between about six hours and about twelve hours or between about six hours and about eight hours, about six, about seven, about eight, about nine, about ten, about eleven, or about twelve consecutive hours, and optionally for less than 24 consecutive hours, e.g., less than twelve, sixteen or eighteen consecutive hours, following each administration. In embodiments, the disclosure includes administering Compound 2, or a pharmaceutically acceptable salt or deuterated form thereof, according to the above methods.

In another embodiment, the disclosure provides a method of increasing the force generation by skeletal muscles including diaphragm of a subject having Duchenne Muscular Dystrophy (DMD), comprising administering a therapeutically effective amount of Compound 1, or a pharmaceutically acceptable salt or deuterated form thereof, including a crystalline form thereof (e.g., co-crystal and polymorph forms), for a treatment period, wherein each treatment period comprises two or more consecutive treatment cycles, wherein each treatment cycle comprises administering Compound 1 or the pharmaceutically acceptable salt or deuterated form thereof to the subject for 3, 4, 5, 6, 7, 8, 9, or 10 consecutive days, followed by an interval of 1, 2, 3, or 4 days wherein Compound 1 or the pharmaceutically acceptable salt or deuterated form thereof is not administered to the subject. In particular embodiments, during each treatment cycle, the subject is administered Compound 1 or a pharmaceutically acceptable salt or deuterated form thereof for at least four consecutive days e.g., four to six days, followed by an interval of at least one day, e.g., one to three days, of not being administered Compound 1 or a pharmaceutically acceptable salt or deuterated form thereof. In particular embodiments, during each treatment cycle, the subject is administered Compound 1 or a pharmaceutically acceptable salt or deuterated form thereof for five consecutive days followed by an interval of two days of not being administered Compound 1 or a pharmaceutically acceptable salt or deuterated form thereof. In particular embodiments, Compound 1 or a pharmaceutically acceptable salt or deuterated form thereof is administered orally. In particular embodiments, Compound 1 or a pharmaceutically acceptable salt or deuterated form thereof, or a crystalline form thereof of Compound 1 is administered at a dose of about 20 mg to about 500 mg or about 50 mg to about 400 mg per day on days of administration. In particular embodiments, Compound 1 or a pharmaceutically acceptable salt or deuterated form thereof, or a crystalline form thereof of Compound 1 is administered at a dose of about 50 mg to about 250 mg per day on days of administration. In particular embodiments, Compound 1 or a pharmaceutically acceptable salt or deuterated form thereof, or a crystalline form thereof of Compound 1 is administered at a dose of about 60 mg to about 240 mg per day on days of administration. In particular embodiments, Compound 1 or a pharmaceutically acceptable salt or deuterated form thereof, or a crystalline form thereof of Compound 1 is administered at a dose of about 100 mg to about 250 mg per day on days of administration. In particular embodiments, Compound 1 or a pharmaceutically acceptable salt or deuterated form thereof, or a crystalline form thereof of Compound 1 is administered at a dose of about 20 mg to about 200 mg per day on days of administration. In particular embodiments, Compound 1 or a pharmaceutically acceptable salt or deuterated form thereof is administered at a dose sufficient to achieve a blood plasma level of greater than about 130 ng/ml for at least six or at least eight consecutive hours, e.g., between about six hours and about twelve hours or between about six hours and about eight hours, about six, about seven, about eight, about nine, about ten, about eleven, or about twelve consecutive hours, and optionally for less than 24 consecutive hours, e.g., less than twelve, sixteen or eighteen consecutive hours, following each administration. In embodiments, the disclosure includes administering Compound 2, or a pharmaceutically acceptable salt or deuterated form thereof, according to the above methods.

In another related embodiment, the disclosure provides a method of increasing the forced vital capacity (FVC) of a subject having DMD, comprising administering a therapeutically effective amount of Compound 1, or a pharmaceutically acceptable salt or deuterated form thereof, or a crystalline form thereof (e.g., a cocrystal or polymorph) for a treatment period, wherein each treatment period comprises two or more consecutive treatment cycles, wherein each treatment cycle comprises administering Compound 1 or the pharmaceutically acceptable salt or deuterated form thereof to the subject for 3, 4, 5, 6, 7, 8, 9, or 10 consecutive days, followed by an interval of 1, 2, 3, or 4 days wherein Compound 1 or the pharmaceutically acceptable salt or deuterated form thereof is not administered to the subject. In particular embodiments, during each treatment cycle, the subject is administered Compound 1 or a pharmaceutically acceptable salt or deuterated form thereof for at least four consecutive days, e.g., four to six days, followed by an interval of at least one day, e.g., one to three days, of not being administered Compound 1 or a pharmaceutically acceptable salt or deuterated form thereof. In particular embodiments, during each treatment cycle, the subject is administered Compound 1 or a pharmaceutically acceptable salt or deuterated form thereof for five consecutive days followed by an interval of two days of not being administered Compound 1 or a pharmaceutically acceptable salt or deuterated form thereof. In particular embodiments, Compound 1 or a pharmaceutically acceptable salt or deuterated form thereof is administered orally. In particular embodiments, Compound 1 or a pharmaceutically acceptable salt or deuterated form thereof, or a crystalline form thereof of Compound 1 is administered at a dose of about 20 mg to about 500 mg or about 50 mg to about 400 mg per day on days of administration. In particular embodiments, Compound 1 or a pharmaceutically acceptable salt or deuterated form thereof, or a crystalline form thereof of Compound 1 is administered at a dose of about 50 mg to about 250 mg per day on days of administration. In particular embodiments, Compound 1 or a pharmaceutically acceptable salt or deuterated form thereof, or a crystalline form thereof of Compound 1 is administered at a dose of about 60 mg to about 240 mg per day on days of administration. In particular embodiments, Compound 1 or a pharmaceutically acceptable salt or deuterated form thereof, or a crystalline form thereof of Compound 1 is administered at a dose of about 100 mg to about 250 mg per day on days of administration. In particular embodiments, Compound 1 or a pharmaceutically acceptable salt or deuterated form thereof, or a crystalline form thereof of Compound 1 is administered at a dose of about 20 mg to about 200 mg per day on days of administration. In particular embodiments, Compound 1 or a pharmaceutically acceptable salt or deuterated form thereof is administered at a dose sufficient to achieve a blood plasma level of greater than about 130 ng/mL for at least six or at least eight consecutive hours, e.g., between about six hours and about twelve hours or between about six hours and about eight hours, about six, about seven, about eight, about nine, about ten, about eleven, or about twelve consecutive hours, and optionally for less than 24 consecutive hours, e.g., less than twelve, sixteen or eighteen consecutive hours, following each administration. In embodiments, the disclosure includes administering Compound 2, or a pharmaceutically acceptable salt or deuterated form thereof, according to the above methods.

In another related embodiment, the disclosure provides a method of increasing the 99th Percentile Maximum Effort in a subject having DMD, comprising administering a therapeutically effective amount of Compound 1, or a pharmaceutically acceptable salt or deuterated form thereof, or a crystal form thereof (e.g., a cocrystal or polymorph) for a treatment period, wherein each treatment period comprises two or more consecutive treatment cycles, wherein each treatment cycle comprises administering Compound 1 or the pharmaceutically acceptable salt or deuterated form thereof to the subject for 3, 4, 5, 6, 7, 8, 9, or 10 consecutive days, followed by an interval of 1, 2, 3, or 4 days wherein Compound 1 or the pharmaceutically acceptable salt or deuterated form thereof is not administered to the subject. In particular embodiments, during each treatment cycle, the subject is administered Compound 1 or a pharmaceutically acceptable salt or deuterated form thereof for at least four consecutive days, e.g., four to six days, followed by an interval of at least one day, e.g., one to three days, of not being administered Compound 1 or a pharmaceutically acceptable salt or deuterated form thereof. In particular embodiments, during each treatment cycle, the subject is administered Compound 1 or a pharmaceutically acceptable salt or deuterated form thereof for five consecutive days followed by an interval of two days of not being administered Compound 1 or a pharmaceutically acceptable salt or deuterated form thereof. In particular embodiments, Compound 1 or a pharmaceutically acceptable salt or deuterated form thereof is administered orally. In particular embodiments, Compound 1 or a pharmaceutically acceptable salt or deuterated form thereof, or a crystalline form thereof of Compound 1 is administered at a dose of about 20 mg to about 500 mg or about 50 mg to about 400 mg per day on days of administration. In particular embodiments, Compound 1 or a pharmaceutically acceptable salt or deuterated form thereof, or a crystalline form thereof of Compound 1 is administered at a dose of about 50 mg to about 250 mg per day on days of administration. In particular embodiments, Compound 1 or a pharmaceutically acceptable salt or deuterated form thereof, or a crystalline form thereof of Compound 1 is administered at a dose of about 60 mg to about 240 mg per day on days of administration. In particular embodiments, Compound 1 or a pharmaceutically acceptable salt or deuterated form thereof, or a crystalline form thereof of Compound 1 is administered at a dose of about 100 mg to about 250 mg per day on days of administration. In particular embodiments, Compound 1 or a pharmaceutically acceptable salt or deuterated form thereof, or a crystalline form thereof of Compound 1 is administered at a dose of about 20 mg to about 200 mg per day on days of administration. In particular embodiments, Compound 1 or a pharmaceutically acceptable salt or deuterated form thereof is administered at a dose sufficient to achieve a blood plasma level of greater than about 130 ng/mL for at least six or at least eight consecutive hours, e.g., between about six hours and about twelve hours or between about six hours and about eight hours, about six, about seven, about eight, about nine, about ten, about eleven, or about twelve consecutive hours, and optionally for less than 24 consecutive hours, e.g., less than twelve, sixteen or eighteen consecutive hours, following each administration. In embodiments, the disclosure includes administering Compound 2, or a pharmaceutically acceptable salt or deuterated form thereof, according to the above methods.

### Compounds

### Compound 1

Compound 1 is an AP2 Associated Kinase 1 (AAK1) inhibitor having the structure:

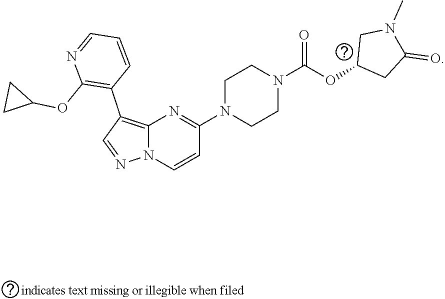 [SMILES: CN1C[C@@H](OC(=O)N2CCN(c3ccn4ncc(-c5cccnc5OC5CC5)c4n3)CC2)CC1=O]

Compound 2 is an AP2 Associated Kinase 1 (AAK1) inhibitor having the structure:

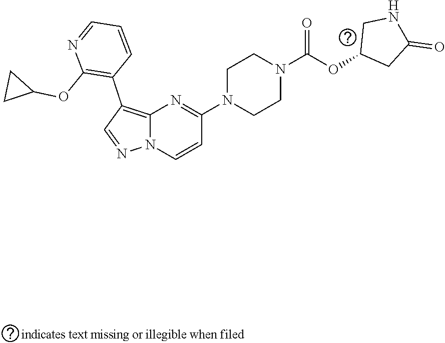 [SMILES: O=C1C[C@H](OC(=O)N2CCN(c3ccn4ncc(-c5cccnc5OC5CC5)c4n3)CC2)CN1]

The synthesis and uses of Compound 1 and Compound 2 are described in International Application No. US2024/045579, which is incorporated by reference in its entirety for all purposes.

The present disclosure provides pharmaceutically acceptable salts, cocrystals, crystalline forms, and polymorph forms of Compound 1 and Compound 2. The present disclosure also provides amorphous forms of Compound 1 and Compound 2 and pharmaceutically salts thereof. Methods of treatment and methods of preparation of the disclosed compounds are also provided herein.

### Compound 1 Oxalate Salt and Oxalic Acid Cocrystal

In some embodiments, the present disclosure provides a pharmaceutically acceptable salt of Compound 1. In some embodiments, the pharmaceutically acceptable salt of Compound 1 is an oxalate salt. The disclosure encompasses all oxalate anions. In some embodiments, the Compound 1 oxalate is crystalline (“Compound 1 Form I oxalate salt”). In some embodiments, Compound 1 and oxalic acid exist as a co-crystal (“Compound 1 Form I oxalic acid cocrystal”). In embodiments, the ratio of Compound 1: oxalic acid in the salt or cocrystal is about 1:1, about 1:2, or about 1:3, as shown in the structure below. In some embodiments, the ratio of Compound 1: oxalic acid in the salt or cocrystal is about 1:1 as determined by ion chromatography (IC).

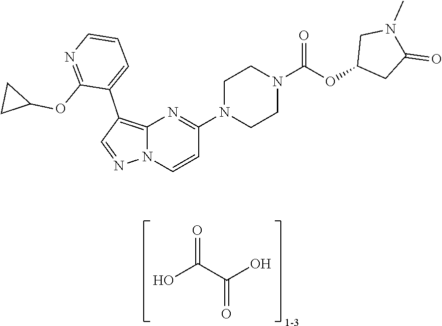

In one embodiment, the present disclosure provides a Compound 1 Form I oxalic acid cocrystal. In some embodiments, the Compound 1 Form I oxalic acid cocrystal exhibits an XRPD pattern comprising 1, 2, 3, 4, or 5 or more peaks at 6.68, 8.43, 10.29, 10.82, 12.28, 14.37, 20.60, 21.78, and 22.45 degrees two-theta with the margin of error of ±0.5; ±0.4; ±0.3; ±0.2; ±0.1; ±0.05; or less. In some embodiments, the Compound 1 Form I oxalic acid cocrystal exhibits an XRPD pattern comprising peaks at 6.68, 8.43, 10.29, 10.82, 12.28, 14.37, 20.60, 21.78, and 22.45 degrees two-theta with the margin of error of ±0.5; ±0.4; ±0.3; ±0.2; ±0.1; ±0.05; or less. In some embodiments, the Compound 1 Form I oxalic acid cocrystal exhibits an XRPD pattern comprising peaks at 10.29, 12.28, and 21.78 degrees two-theta with the margin of error of ±0.5; ±0.4; ±0.3; ±0.2; ±0.1; ±0.05; or less. In some embodiments, the XRPD peaks have a margin of error of ±0.2. In some embodiments, the XRPD peaks have a margin of error of +0.1. In yet another embodiment, the Compound 1 Form I oxalic acid cocrystal exhibits an XRPD comprising peaks shown in Table 1 below.

TABLE 1
XRPD Table of Compound 1 Form I oxalate

| | Relative |
|---|---|
| 2-Theta | Intensity % |
| 6.68 | 10.1 |
| 8.43 | 17.9 |
| 10.29 | 100 |
| 10.82 | 16.6 |
| 12.28 | 79.0 |
| 14.37 | 10.7 |
| 20.60 | 11.0 |
| 21.78 | 25.4 |
| 22.45 | 11.6 |

In some embodiments, the Compound 1 Form I oxalic acid cocrystal exhibits a DSC thermogram comprising an initial sharp endotherm at 169° C. with the margin of error of ±10.0; ±5.0; ±4.0; ±3.0; ±2.0; ±1.0; or less. In some embodiments, the Compound 1 Form I oxalic acid cocrystal exhibits a DSC thermogram comprising two additional endotherms with onset temperatures of 180° C. and 245° C. with the margin of error of ±10.0; ±5.0; ±4.0; ±3.0; ±2.0; ±1.0; or less. In some embodiments, the TGA thermogram of the Compound 1 Form I oxalic acid cocrystal exhibits a weight loss of about 15-25% that onsets at a temperature of 179±5° C. In other embodiments, the TGA thermogram of the Compound 1 Form I oxalic acid cocrystal exhibits a weight loss of about 18.2% that onsets at a temperature of about 179° C. In some embodiments, the TGA thermogram of the Compound 1 Form I oxalic acid cocrystal exhibits a second weight loss of about 15-25% that onsets at a temperature of 241±5° C. In further embodiments, the TGA thermogram of the Compound 1 Form I oxalic acid cocrystal exhibits a second weight loss of about 18.9% that onsets at a temperature of about 241° C.

The Compound 1 salts and cocrystals disclosed herein may contain minor impurities that result, e.g., from the synthesis methods or the partial conversion of one crystalline form to another. Impurities may also be in the form of residual solvent or water that is present outside of the lattice.

In one embodiment, Compound 1 has a purity of at least about 75%, at least about 80%, at least about 85%, at least about 90%, at least about 95%, at least about 97%, at least about 98%, or at least about 99%.

In one embodiment, Compound 1 has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, Compound 1 has a purity of about 75% to about 99%. In one embodiment, Compound 1 has a purity of about 80% to about 99%. In one embodiment, the Compound 1 oxalate has a purity of about 85% to about 99%. In one embodiment, the Compound 1 oxalate has a purity of about 90% to about 99%. In one embodiment, the Compound 1 oxalate has a purity of about 95% to about 99%. In one embodiment, the Compound 1 oxalate has a purity of about 97.5% to about 99%.

In one embodiment, the specific solid form of the Compound 1 with high purity is Form I oxalic acid cocrystal. In some embodiments, the Compound 1 Form I oxalic acid cocrystal is prepared according to the procedure described in Example 8.

In some embodiments, the present disclosure provides a chloride salt or cocrystal of Compound 1 (“Compound 1 chloride”). In some embodiments, the present disclosure provides a crystalline form of Compound 1 chloride. In some embodiments, the crystalline form is a Compound 1 salt (“Compound 1 Form I chloride salt”) or a Compound 1 cocrystal (“Compound 1 Form I hydrochloride cocrystal”). In some embodiments, the crystalline form is a polymorph form. In some embodiments, the Compound 1 chloride is amorphous.

In one embodiment, the present disclosure provides Compound 1 Form I chloride. In some embodiments, the Compound 1 Form I chloride exhibits an XRPD pattern comprising 1, 2, 3, 4, or 5 or more peaks at 7.37, 8.97, 11.09, 13.89, 14.83, 16.28, 18.06, 20.44, 20.53, 24.84, 25.18, 25.22, and 26.14 degrees two-theta with the margin of error of =0.5; +0.4; +0.3; +0.2; +0.1; +0.05; or less. In some embodiments, the Compound 1 Form I chloride exhibits an XRPD pattern comprising peaks at 7.37, 8.97, 11.09, 13.89, 14.83, 16.28, 18.06, 20.44, 20.53, 24.84, 25.18, 25.22, and 26.14 degrees two-theta with the margin of error of +0.5; +0.4; +0.3; +0.2; +0.1; +0.05; or less. In some embodiments, the Compound 1 Form I chloride exhibits an XRPD pattern comprising 1, 2, 3, 4, or 5 or more peaks at 8.97, 11.09, 13.89, 14.83, 20.53, 25.18, 25.22, and 26.14 degrees two-theta with the margin of error of ±0.5; ±0.4; ±0.3; ±0.2; ±0.1; ±0.05; or less. In some embodiments, the XRPD peaks have a margin of error of ±0.2. In some embodiments, the XRPD peaks have a margin of error of ±0.1. In yet another embodiment, the Compound 1 Form I chloride exhibits an XRPD comprising peaks shown in Table 2 below.

TABLE 2
XRPD Table of Compound 1 Form I chloride.

| | Relative |
|---|---|
| 2-Theta | Intensity % |
| 7.37 | 11.4 |
| 8.97 | 37.3 |
| 11.09 | 100.0 |
| 13.89 | 35.2 |
| 14.83 | 20.1 |
| 16.28 | 13.0 |
| 18.06 | 14.1 |
| 20.44 | 17.3 |
| 20.53 | 22.5 |
| 24.84 | 15.2 |
| 25.18 | 39.7 |
| 25.22 | 38.1 |
| 26.14 | 19.7 |

In some embodiments, the Compound 1 Form I chloride exhibits a DSC thermogram comprising an initial broad endotherm at 42° C. with the margin of error of ±2.5; about ±2.0; about ±1.5; about ±1.0; about ±0.5; or less. In some embodiments, the Compound 1 Form I chloride exhibits a DSC thermogram comprising an additional endotherm centered at 131° C. with the margin of error of ±10.0; ±5.0; ±4.0; ±3.0; ±2.0; ±1.0; or less. In further embodiments, the Compound 1 Form I chloride exhibits a DSC thermogram comprising an additional endotherm that onsets at a temperature of 192° C. having a margin of error of ±10.0; ±5.0; ±4.0; ±3.0; ±2.0; ±1.0; or less. In some embodiments, the TGA thermogram of the Compound 1 Form I chloride exhibits a weight loss of about 1-5% from 25° C. to 180° C. In other embodiments, the TGA thermogram of the Compound 1 Form I chloride exhibits a weight loss of about 1.7% from 25° C. to 179° C. In some embodiments, the TGA thermogram of the Compound 1 Form I chloride exhibits additional weight loss events of about 5-10% centered on a temperature of 187±5° C.° C. and about 15-25% centered on a temperature 249±5° C. In further embodiments, the TGA thermogram of the Compound 1 Form I chloride exhibits additional weight loss events of about 7.2% centered on a temperature of about 187° C. and about 20.1% centered on a temperature of about 249° C.

In one embodiment, Compound 1 has a purity of at least about 75%, at least about 80%, at least about 85%, at least about 90%, at least about 95%, at least about 97%, at least about 98%, or at least about 99%.

In one embodiment, Compound 1 has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, Compound 1 has a purity of about 75% to about 99%. In one embodiment, Compound 1 has a purity of about 80% to about 99%. In one embodiment, Compound 1 has a purity of about 85% to about 99%. In one embodiment, Compound 1 has a purity of about 90% to about 99%. In one embodiment, Compound 1 has a purity of about 95% to about 99%. In one embodiment, Compound 1 has a purity of about 97.5% to about 99%.

In one embodiment, the specific solid form of the Compound 1 with high purity is crystalline Form I chloride. In some embodiments, the Compound 1 Form I chloride is prepared according to the procedure described in Example 9.

In some embodiments, the present disclosure provides a phosphate salt or cocrystal of Compound 1 (“Compound 1 phosphate”). In some embodiments, the present disclosure provides a crystalline form of Compound 1 In some embodiments, the crystalline form is a Compound 1 salt (“Compound 1 Form I phosphate salt”) or a Compound 1 cocrystal (“Compound 1 Form I phosphoric acid cocrystal”). In some embodiments, the Compound 1 phosphate is a amorphous.

In one embodiment, the present disclosure provides Compound 1 Form I phosphate. In some embodiments, the Compound 1 Form I phosphate exhibits an XRPD pattern comprising 1, 2, 3, 4, or 5 or more peaks at 3.66, 6.26, 7.34, 11.02, 12.56, 14.15, 14.39, 14.99, 15.32, 16.19, 17.72, 18.38, 18.86, 19.50, 19.77, 20.76, 21.08, 21.29, 22.08, 22.33, 22.76, 22.83, 23.14, 23.39, 23.68, 24.14, 24.52, 26.64, and 28.76 degrees two-theta with the margin of error of ±0.5; ±0.4; ±0.3; ±0.2; ±0.1; ±0.05; or less. In some embodiments, the Compound 1 Form I phosphate exhibits an XRPD pattern comprising peaks at 3.66, 6.26, 7.34, 11.02, 12.56, 14.15, 14.39, 14.99, 15.32, 16.19, 17.72, 18.38, 18.86, 19.50, 19.77, 20.76, 21.08, 21.29, 22.08, 22.33, 22.76, 22.83, 23.14, 23.39, 23.68, 24.14, 24.52, 26.64, and 28.76 degrees two-theta with the margin of error of ±0.5; ±0.4; ±0.3; ±0.2; ±0.1; ±0.05; or less. In some embodiments, the Compound 1 Form I phosphate exhibits an XRPD pattern comprising peaks at 7.34, 11.02, 17.72, 18.38, 18.86, 19.50, 19.77, 20.76, 21.29, 22.08, 22.76, 22.83, 23.14, 23.39, 23.68, 24.14, and 24.52 degrees two-theta with the margin of error of ±0.5; ±0.4; ±0.3; ±0.2; ±0.1; ±0.05; or less. In some embodiments, the XRPD peaks have a margin of error of ±0.2. In some embodiments, the XRPD peaks have a margin of error of ±0.1. In yet another embodiment, the Compound 1 Form I phosphate exhibits an XRPD comprising peaks shown in Table 3 below.

TABLE 3
XRPD Table of Compound 1 Form I phosphate.

| | Relative |
|---|---|
| 2-Theta | Intensity % |
| 3.66 | 13.7 |
| 6.26 | 13.9 |
| 7.34 | 45.4 |
| 11.02 | 100.0 |
| 12.56 | 12.3 |
| 14.15 | 12.3 |
| 14.39 | 11.0 |
| 14.99 | 16.1 |
| 15.32 | 13.5 |
| 16.19 | 10.5 |
| 17.72 | 35.8 |
| 18.38 | 49.9 |
| 18.86 | 24.4 |
| 19.50 | 23.0 |
| 19.77 | 38.5 |
| 20.76 | 22.4 |
| 21.08 | 17.8 |
| 21.29 | 23.0 |
| 22.08 | 20.7 |
| 22.33 | 14.9 |
| 22.76 | 31.8 |
| 22.83 | 47.1 |
| 23.14 | 41.1 |
| 23.39 | 28.7 |
| 23.68 | 21.8 |
| 24.14 | 32.1 |
| 24.52 | 36.9 |
| 26.64 | 15.8 |
| 28.76 | 13.1 |

In some embodiments, the Compound 1 Form I phosphate exhibits a DSC thermogram comprising a first small endotherm that onsets at a temperature of 40° C. and a second broad endotherm that onsets at a temperature of 78° C. having a margin of error of ±10.0; ±5.0; ±4.0; ±3.0; ±2.0; ±1.0; or less. In some embodiments, the Compound 1 Form I phosphate exhibits a DSC thermogram comprising an additional endotherm that onsets at a temperature of 154° C. with the margin of error of ±10.0; ±5.0; ±4.0; ±3.0; ±2.0; =1.0; or less. In further embodiments, the Compound 1 Form I phosphate exhibits a DSC thermogram comprising an additional endotherm that onsets at a temperature of 221° C. having a margin of error of =10.0; ±5.0; ±4.0; ±3.0; ±2.0; ±1.0; or less. In other embodiments, the TGA thermogram of the Compound 1 Form I phosphate exhibits a weight loss of about 1-10% that onsets at a temperature of 73±5° C. In other embodiments, the TGA thermogram of the Compound 1 Form I phosphate exhibits a weight loss of about 5.72% that onsets at a temperature of 73° C. In some embodiments, the TGA thermogram of the Compound 1 Form I phosphate exhibits an additional weight loss of about 5-15% that onsets at a temperature of 194±5° C. In further embodiments, the TGA thermogram of the Compound 1 Form I phosphate exhibits an additional weight loss of 9.46% that onsets at a temperature of about 194° C.

In some embodiments, the Compound 1 Form I phosphate exhibits a mass increase of about 5-15% due to moisture intake at 80-90% relative humidity (RH). In some embodiments, the Compound 1 Form I phosphate exhibits a mass increase of about 5-10% due to moisture intake at about 90% RH. In some embodiments, the Compound 1 Form I phosphate exhibits a mass increase of about 7.2% due to moisture intake at about 90% RH. In some embodiments, the Compound 1 Form I phosphate exhibits a mass increase of about 2-10% due to moisture intake at 50-60% RH. In some embodiments, the Compound 1 Form I phosphate exhibits a mass increase of about 3-7% due to moisture intake at about 50% RH. In some embodiments, the Compound 1 Form I phosphate exhibits a mass increase of about 5.1% due to moisture intake at about 50% RH. In some embodiments, the Compound 1 Form I phosphate exhibits a mass loss on drying of about 10% or less at about 0% RH. In some embodiments, the Compound 1 Form I phosphate exhibits a mass loss on drying of about 3-7% at about 0% RH. In some embodiments, the Compound 1 Form I phosphate exhibits a mass loss on drying of about 5% at about 0% RH.

In one embodiment, Compound 1 has a purity of at least about 75%, at least about 80%, at least about 85%, at least about 90%, at least about 95%, at least about 97%, at least about 98%, or at least about 99%.

In one embodiment, Compound 1 has a purity of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, or about 90%. In one embodiment, Compound 1 has a purity of about 75% to about 99%. In one embodiment, Compound 1 has a purity of about 80% to about 99%. In one embodiment, Compound 1 has a purity of about 85% to about 99%. In one embodiment, Compound 1 has a purity of about 90% to about 99%. In one embodiment, Compound 1 has a purity of about 95% to about 99%. In one embodiment, Compound 1 has a purity of about 97.5% to about 99%.

In one embodiment, the specific solid form of the Compound 1 phosphate with high purity is crystalline Form I phosphate. In some embodiments, the Compound 1 Form I phosphate is prepared according to the procedure described in Example 10.

### Treatment Periods and Treatment Cycles

The methods described herein comprise administering Compound 1 or a pharmaceutically acceptable salt or deuterated form thereof, or a crystalline or polymorph form of Compound 1 to a subject for a treatment period. In some embodiments, the treatment period is at least 1, at least 2, at least 3, at least 4, at least 5, or at least 6 months. In some embodiments, the treatment period spans the entire remaining lifetime of the subject or a substantial portion thereof.

A treatment period may comprise treatment cycles. In some embodiments, a treatment period comprises at least 2, at least 3, at least 4, at least 6, at least 8, or at least 12 treatment cycles. In some embodiments, the treatment period comprises at least 4 treatment cycles.

A treatment cycle comprises at least 1 day wherein Compound 1 or a pharmaceutically acceptable salt or deuterated form thereof, or a crystalline form thereof is administered to the subject and at least 1 day wherein Compound 1 or a pharmaceutically acceptable salt or deuterated form thereof, or a crystalline form thereof is not administered to the subject. In some embodiments, the treatment cycle is at least 3, at least 4, at least 5, at least 6, at least 7, at least 8, at least 9, at least 10, at least 11, at least 12, at least 13, or at least 14 days in length, and in some embodiments, the treatment cycle is at most 5, at most 6, at most 7, at most 8, at most 9, at most 10, at most 11, at most 12, at most 13, or at most 14 days in length. In some embodiments, the treatment cycle is 7 days in length.

In some embodiments, a treatment cycle comprises administering Compound 1 or a pharmaceutically acceptable salt or deuterated form thereof, or a crystalline form thereof to the subject for 3, 4, 5, 6, 7, 8, 9, 10, 11, or 12 days within a treatment cycle. In some embodiments, a treatment cycle comprises administering Compound 1 or a pharmaceutically acceptable salt or deuterated form thereof, or a crystalline form thereof to the subject for 3, 4, 5, 6, 7, 8, 9, or 10 consecutive days, followed by an interval of 1, 2, 3, or 4 days wherein Compound 1 or the pharmaceutically acceptable salt or deuterated form thereof, or a crystalline form thereof is not administered to the subject. In some embodiments, the treatment cycle comprises administering Compound 1 or a pharmaceutically acceptable salt or deuterated form thereof, or a crystalline form thereof to the subject for 3 consecutive days. In some embodiments, the treatment cycle comprises administering Compound 1 or a pharmaceutically acceptable salt or deuterated form thereof, or a crystalline form thereof to the subject for 4 consecutive days. In some embodiments, the treatment cycle comprises administering Compound 1 or a pharmaceutically acceptable salt or deuterated form thereof, or a crystalline form thereof to the subject for 5 consecutive days. In some embodiments, the treatment cycle comprises administering Compound 1 or a pharmaceutically acceptable salt or deuterated form thereof, or a crystalline form thereof to the subject for 6 consecutive days. In some embodiments, the treatment cycle comprises 1 day wherein Compound 1 or the pharmaceutically acceptable salt or deuterated form thereof, or a crystalline form thereof is not administered to the subject. In some embodiments, the treatment cycle comprises 2 days wherein Compound 1 or the pharmaceutically acceptable salt or deuterated form thereof, or a crystalline form thereof is not administered to the subject. In some embodiments, the treatment cycle comprises 3 days wherein Compound 1 or the pharmaceutically acceptable salt or deuterated form thereof is not administered to the subject. In some embodiments, the treatment cycle comprises 4 days wherein Compound 1 or the pharmaceutically acceptable salt or deuterated form thereof, or a crystalline form thereof is not administered to the subject.

In some embodiments, the treatment cycle comprises administering Compound 1 or a pharmaceutically acceptable salt or deuterated form thereof to the subject for 3 consecutive days, followed by an interval of 4 days wherein Compound 1 or the pharmaceutically acceptable salt or deuterated form thereof is not administered to the subject. In some embodiments, the treatment cycle comprises administering Compound 1 or a pharmaceutically acceptable salt or deuterated form thereof to the subject for 4 consecutive days, followed by an interval of 3 days wherein Compound 1 or the pharmaceutically acceptable salt or deuterated form thereof is not administered to the subject. In some embodiments, the treatment cycle comprises administering Compound 1 or a pharmaceutically acceptable salt or deuterated form thereof Compound 1 to the subject for 5 consecutive days, followed by an interval of 2 days wherein Compound 1 or the pharmaceutically acceptable salt or deuterated form thereof, or a crystalline or polymorph form of Compound 1 is not administered to the subject. In some embodiments, the treatment cycle comprises administering Compound 1 or a pharmaceutically acceptable salt or deuterated form thereof, to the subject for 6 consecutive days, followed by an interval of 1 day wherein Compound 1 or the pharmaceutically acceptable salt or deuterated form thereof is not administered to the subject.

In certain embodiments, the treatment period comprises at least one 7-day treatment cycle, wherein Compound 1 or the pharmaceutically acceptable salt or deuterated form thereof, is administered for 5 days of each 7-day treatment cycle and not administered for 2 days of each 7-day treatment cycle. In certain embodiments, the treatment period comprises at least one 7-day treatment cycle, wherein Compound 1 or the pharmaceutically acceptable salt or deuterated form thereof is administered for 5 consecutive days of each 7-day treatment cycle and not administered for 2 days of each 7-day treatment cycle. In some embodiments, the treatment period comprises at least one 7-day treatment cycle, wherein Compound 1 or the pharmaceutically acceptable salt or deuterated form thereof is administered for 4 or more days of each 7-day treatment cycle and not administered for 1 or more day of each 7-day treatment cycle. In some embodiments, the treatment period comprises at least one 7-day treatment cycle, wherein Compound 1 or the pharmaceutically acceptable salt or deuterated form thereof is administered for 5 or more days of each 7-day treatment cycle and not administered for 1 or more day of each 7-day treatment cycle. In some embodiments, the treatment period comprises at least one 7-day treatment cycle, wherein Compound 1 or the pharmaceutically acceptable salt or deuterated form thereof is administered for 5 or more days of each 7-day treatment cycle and not administered for 1 or more day of each 7-day treatment cycle.

In some embodiments, each of the treatment cycles within a treatment period is the same. As an example, a treatment period of at least 1 month may comprise four 7-day treatment cycles, each of which comprises administering Compound 1 or a pharmaceutically acceptable salt or deuterated form thereof to the subject for 5 consecutive days, followed by an interval of 2 days wherein Compound 1 or the pharmaceutically acceptable salt or deuterated form thereof is not administered to the subject. In some embodiments, at least two treatment cycles within a treatment period are different. As an example, a treatment period of at least 1 month may comprise four 7-day treatment cycles, with one of the four cycles comprising administering Compound 1 or a pharmaceutically acceptable salt or deuterated form thereof to the subject for 5 consecutive days, followed by an interval of 2 days wherein Compound 1 or the pharmaceutically acceptable salt or deuterated form thereof is not administered to the subject and another one of the four cycles comprising administering Compound 1 or a pharmaceutically acceptable salt or deuterated form thereof to the subject for 4 consecutive days, followed by an interval of 3 days wherein Compound 1 or the pharmaceutically acceptable salt or deuterated form thereof is not administered to the subject.

In some embodiments, administration of Compound 1 or a pharmaceutically acceptable salt or deuterated form thereof is used to establish its effect in a subject. The establishment of the effect in a subject may be measured by determining the blood plasma level of Compound 1 or the pharmaceutically acceptable salt or deuterated form thereof in the subject. Determining whether the effect is established may also involve measuring the IC50 of Compound 1 and the length of time for which the IC50 level is maintained in blood plasma. Once the effect is established, the subject may stop taking the drug for an interval of at least 1 day. After said interval, the subject may resume taking the drug again for a period of time such that the effect is observed again, for example, until the IC50 is again maintained in the blood plasma for a predefined length of time.

In some embodiments of the methods described herein, the blood plasma level of Compound 1 or a pharmaceutically acceptable salt or deuterated form thereof in the subject is above a minimum effective level for a period of time during the treatment period immediately prior to the interval wherein Compound 1 or the pharmaceutically acceptable salt or deuterated form thereof, or a crystalline or polymorph form of Compound 1 is not administered, and during said interval, the blood plasma level is below or falls below the minimum effective level. In some embodiments, the minimum effective blood plasma level is 10-100 ng/ml, 10-200 ng/mL, 50-150 ng/mL, about 50 ng, about 100 ng, about 120 ng, about 130 ng, about 140 ng, about 150 ng, or about 200 ng. In some embodiments, the minimum effective blood plasma level is about 10 ng/mL. In some embodiments, the minimum effective blood plasma level is about 50 ng/ml. In some embodiments, the minimum effective blood plasma level is about 100 ng/mL plasma drug levels. In some embodiments, the blood plasma level is above the minimal effective amount for about 6-8 hours. In some embodiments, the minimum effective level is the IC50 of compound being administered.

In some embodiments of the methods described herein, the blood plasma level of Compound 1 or a pharmaceutically acceptable salt or deuterated form thereof, or a crystalline or polymorph form of Compound 1 in the subject is maintained for at least 6 hours, and optionally less than above the IC50 for Compound 1 or a pharmaceutically acceptable salt or deuterated form thereof prior to the interval wherein Compound 1 or the pharmaceutically acceptable salt or deuterated form thereof, or the crystalline or polymorph form of Compound 1 is not administered, and during said interval, the blood plasma level falls below said IC50. In certain embodiments, the blood plasma level is above the IC50 for at least 6 hours. In certain embodiments, the blood plasma level is above the IC50 for 6-8 hours. The IC50 may be predetermined.

IC50 of the compounds disclosed herein against AAK1 (amino acids T27-A365) may be determined as follows. AAK1 protein (amino acids T27-A365) is dissolved at 20 nM in 1× enzyme solution (50 mM HEPES pH7.5, 10 mM MgCl2, 1 mM DTT, 1% glycerol, 0.5 mM EGTA, 0.01% Briji35, 0.02% BSA) and 12.5 ul is incubated with 250 nl DMSO or 250 nl compound in DMSO at 25° C. for 15 min. The reaction is started by addition of 12.5 μl of 30 μM AQT0759 (Assayquant, CSKS-AQT0759B) and 70 μM ATP which are diluted in 1× substrate dilution buffer (50 mM HEPES pH7.5, 10 mM MgCl2, 1 mM DTT, 1% glycerol, 0.5 mM EGTA, 0.01% Briji35). The rate of product formation (fluorescence signal with Excitation at 360 nm and Emission at 486 nm, Molecular Devices SpectraMax Paradigm) is measured every 2 minutes for 120 minutes at 25° C. and the RFU data is analyzed by linear regression. For IC50 determination, rates normalized relative to uninhibited controls are plotted against compound concentration and fitted using a 4 parameter non-linear regression curve fit (Y=Bottom+ ((Top−Bottom)/(1+ ((IC50/X)\hat{ }Slope))), XLfit Model 205). IC50 is determined in nM units, which are converted to ng/mL units by multiplying the IC50 nM value by 4.3.

### Dose

In some embodiments of the methods described herein, Compound 1 or a pharmaceutically acceptable salts or deuterated forms thereof, is administered once daily. In other embodiments, Compound 1 or a pharmaceutically acceptable salts or deuterated forms thereof, is administered at least twice daily. In some embodiments, Compound 1 or a pharmaceutically acceptable salt thereof to a subject is administered twice daily.

In some embodiments, 10 mg-1000 mg of Compound 1 or Compound 2, or an equivalent dose of a pharmaceutically acceptable salt or deuterated form thereof is administered to the subject daily. In some embodiments, 10 mg-400 mg of Compound 1 or an equivalent dose of a pharmaceutically acceptable salt or deuterated form thereof, or a crystalline or polymorph form of Compound 1 is administered to the subject daily. In some embodiments, about 10 mg-100 mg of Compound 1 or an equivalent dose of a pharmaceutically acceptable salt or deuterated form thereof is administered to the subject daily. When reference is made to “administered daily,” it means on those days of the dosing schedule where the compound is administered. In some embodiments, about 20 mg-200 mg of Compound 1 or an equivalent dose of a pharmaceutically acceptable salt or deuterated form thereof is administered to the subject daily. In some embodiments, about 50 mg-150 mg of Compound 1 or an equivalent dose of a pharmaceutically acceptable salt or deuterated form thereof is administered to the subject daily. In some embodiments, about 60 mg-120 mg of Compound 1 or an equivalent dose of a pharmaceutically acceptable salt or deuterated form thereof is administered to the subject daily. In some embodiments, about 120 mg-240 mg of Compound 1 or an equivalent dose of a pharmaceutically acceptable salt or deuterated form thereof, is administered to the subject daily. In some embodiments, about 120 mg-300 mg of Compound 1 or an equivalent dose of a pharmaceutically acceptable salt or deuterated form thereof is administered to the subject daily. In some embodiments, about 300 mg-400 mg of Compound 1 or an equivalent dose of a pharmaceutically acceptable salt or deuterated form thereof, or a crystalline or polymorph form of Compound 1 is administered to the subject daily. In embodiments, the disclosure includes administering Compound 2, or a pharmaceutically acceptable salt or deuterated form thereof, according to the above methods.

In certain embodiments, about 10 mg of Compound 1 or an equivalent dose of a pharmaceutically acceptable salt or deuterated form thereof is administered to the subject daily. In certain embodiments, about 50 mg of Compound 1 or an equivalent dose of a pharmaceutically acceptable salt or deuterated form thereof is administered to the subject daily. In certain embodiments, about 60 mg of Compound 1 or an equivalent dose of a pharmaceutically acceptable salt or deuterated form thereof is administered to the subject daily. In certain embodiments, about 120 mg of Compound 1 or an equivalent dose of a pharmaceutically acceptable salt or deuterated form thereof is administered to the subject daily. In certain embodiments, about 150 mg of Compound 1 or an equivalent dose of a pharmaceutically acceptable salt or deuterated form thereof is administered to the subject daily. In certain embodiments, about 180 mg of Compound 1 or an equivalent dose of a pharmaceutically acceptable salt or deuterated form thereof is administered to the subject daily. In certain embodiments, about 240 mg of Compound 1 or an equivalent dose of a pharmaceutically acceptable salt or deuterated form thereof is administered to the subject daily. In certain embodiments, about 300 mg of Compound 1 or an equivalent dose of a pharmaceutically acceptable salt or deuterated form thereof, is administered to the subject daily. In certain embodiments, about 400 mg of Compound 1 or an equivalent dose of a pharmaceutically acceptable salt or deuterated form thereof, is administered to the subject daily. In embodiments, the disclosure includes administering Compound 2, or a pharmaceutically acceptable salt or deuterated form thereof, according to the above methods.

### Route of Administration

In embodiments, the methods described herein comprises administering to a subject Compound 1 or a pharmaceutically acceptable salt or deuterated form thereof enterally or parenterally. In some embodiments of the present methods, Compound 1 or a pharmaceutically acceptable salt thereof is administered to the subject systemically or locally, optionally at a site of tissue damage or injury. Compound 1 or a pharmaceutically acceptable salt thereof may be administered to the subject systematically. In some embodiments, Compound 1 or a pharmaceutically acceptable salt or deuterated form thereof is administered enterally. In certain embodiments, the route of administration is oral administration (per os; PO). In some embodiments, Compound 1 or a pharmaceutically acceptable salt or deuterated form thereof is administered parenterally. Examples of parenteral administrations include, but are not limited to, intravenously, intramuscularly, intraperitoneally, or subcutaneously. In embodiments, the disclosure includes administering Compound 2, or a pharmaceutically acceptable salt or deuterated form thereof, according to the above methods.

### Pharmaceutical Compositions

In some embodiments, Compound 1 or a pharmaceutically acceptable salt or deuterated form thereof, or a crystalline or polymorph form of Compound 1 may be formulated in a pharmaceutical composition comprising one or more pharmaceutically acceptable excipients and/or adjuvants. The pharmaceutical composition may be a liquid or solid dosage form. In embodiments, the disclosure includes formulating Compound 2, or a pharmaceutically acceptable salt or deuterated form thereof, in a pharmaceutical composition comprising one or more pharmaceutically acceptable excipients and/or adjuvants.

The pharmaceutically acceptable excipients and adjuvants can be added to the composition or formulation for a variety of purposes. In some embodiments, the excipient is a binder, and/or diluent. In some embodiments, the excipient is a pharmaceutically acceptable carrier. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

### Subjects

The methods described herein comprises administering to a subject Compound 1 or a pharmaceutically acceptable salt or deuterated form thereof, or a crystalline or polymorph form of Compound 1. In some embodiments, the subject is a mammal. In specific embodiments, the subject is a human. In one embodiment, the subject is a human who has DMD. In some embodiments, the subject is ambulatory. In some embodiments, the subject is non-ambulatory. In some embodiments, the subject is a pediatric patient. In some embodiments, the subject is a pediatric patient between 4 and 12 years old, or any value or subrange therein. In certain embodiments, the subject is 4, 5 or 6 years old. In certain embodiments, the subject is 7, 8, or 9 years old. In certain embodiments, the subject is 10, 11, or 12 years old. In embodiments, the disclosure includes administering Compound 2, or a pharmaceutically acceptable salt or deuterated form thereof, according to the above methods.

In some embodiments, the subject is 4, 5, or 6 years old and Compound 1 or a pharmaceutically acceptable salt or cocrystal thereof, or deuterated form thereof, is administered at a dose of about 30 mg to about 120 mg, e.g., about 50 mg to about 60 mg, on each day of administration. In some embodiments, the subject is 4, 5, or 6 years old and Compound 1 or a pharmaceutically acceptable salt or cocrystal thereof, or deuterated form thereof, is administered at a dose of about 30 mg, about 40 mg, about 50 mg, about 60 mg, or about 120 mg, on each day of administration. In certain embodiments, the subject is 4, 5, or 6 years old and Compound 1 or a pharmaceutically acceptable salt or cocrystal thereof, or deuterated form thereof, is administered at a dose of about 50 mg or about 60 mg on each day of administration. In embodiments, the disclosure includes administering Compound 2, or a pharmaceutically acceptable salt or deuterated form thereof, according to the above methods.

In some embodiments, the subject is 7, 8, 9, 10, 11, or 12 years old and Compound 1 or a pharmaceutically acceptable salt or cocrystal thereof, or deuterated form thereof, is administered at a dose of about 30 mg to about 120 mg per day, e.g., 60 mg to about 120 mg per day, on each day of administration. In some embodiments, the subject is 7, 8, 9, 10, 11, or 12 years old and Compound 1 or a pharmaceutically acceptable salt or cocrystal thereof, or deuterated form thereof, is administered at a dose of about 60 mg, about 100 mg, or about 120 mg on each day of administration. In certain embodiments, the subject is 7, 8, 9, 10, 11, or 12 years old and Compound 1 or a pharmaceutically acceptable salt or cocrystal thereof, or deuterated form thereof, is administered at a dose of about 60 mg or about 100 mg on each day of administration. In embodiments, the disclosure includes administering Compound 2, or a pharmaceutically acceptable salt or deuterated form thereof, according to the above methods.

In some embodiments, the subject is an adult. In some embodiments, the subject is at least 18 years old.

In some embodiments, the subject is an adult. In some embodiments, the subject has skeletal stem cells present in the body.

The methods described herein may increase force generation by muscles, such as skeletal muscles, in a subject. The methods described herein may also increase forced vital capacity (FVC) and/or the 99th Percentile Maximum Effort in a subject. Force generation by muscles, FVC, and 99th Percentile Maximum Effort can be determined by methods known in the arts. For example, force generation can be measured by upper extremity dynamometry, FVC can be measured by spirometry, and the 99th Percentile Maximum Effort can be measured by a wearable monitoring device, such as a Syde wearable device.

For ambulant patients affected by Duchenne muscular dystrophy (DMD), the EMA has recently qualified the first digital outcome as a primary endpoint in clinical trials: the stride velocity 95th percentile (SV95C). In certain embodiments, efficacy is measured according to the stride velocity 95^{th} centile as a primary endpoint. See, e.g., Servais L, Yen K, Guridi M, Lukawy J, Vissière D, Strijbos P. Stride Velocity 95th Centile: Insights into Gaining Regulatory Qualification of the First Wearable-Derived Digital Endpoint for use in Duchenne Muscular Dystrophy Trials. J Neuromuscul Dis. 2022; 9 (2): 335-346. doi: 10.3233/JND-210743. PMID: 34958044; PMCID: PMC9028650.

A similar outcome (99th Percentile Maximum Effort) may be determined by measuring the motor function of the upper limbs in non-ambulant patients affected with DMD and spinal muscular atrophy (SMA). Patients wear two magneto-inertial measurement units, one on their wrist and one on their wheelchair. The digital outcome of interest quantifies the gestures where the patients develop the maximum energy with their upper limb, during the monthly recording period. It is called the 99th percentile of the total effort (TE99C).

99th Percentile Maximum Effort and stride velocity 95^{th} centile may be measured using a wearable device, e.g., the Syde device (Sysnav Healthcare, Saint-Marcel, France). The Syde wearable device includes a data acquisition system, which is itself subdivided into 2 parts: 1) two recording wearables worn by the participant, including identical sensors (accelerometers, gyrometers, magnetometers, barometer) calibrated after production and regularly throughout the lifetime of the device; and 2) a docking station, located in the participant's home, that retrieves data from the sensors, recharges the batteries of the wearables, and transmits the data to the software platform. It also includes the software platform for data storage and monitoring by a project manager, and the Analysis software for computing statistical variables on the recordings by a trained analyst.

### Kits

Also provided herein are kits based on Compound 1 or a pharmaceutically acceptable salt or deuterated form thereof, or a crystalline or polymorph form of Compound 1. Also provided herein are kits based on Compound 2 or a pharmaceutically acceptable salt or deuterated form thereof, or a crystalline or polymorph form of Compound 2. Such a kit is particularly suitable for use in the methods as described above.

In some embodiments, the kits described herein comprise at least 3 compound-containing units and optionally at least 1 compound-free unit, wherein each of said compound-containing units comprises Compound 1, or a pharmaceutically acceptable salt or deuterated form thereof, or a crystalline or polymorph form of Compound 1, and each of said compound-free unit does not comprise Compound 1 or the pharmaceutically acceptable salt or deuterated form thereof, or a crystalline or polymorph form of Compound 1. In certain embodiments, the kits described herein comprise 3 compound-containing units and optionally 4 compound-free units. In certain embodiments, the kits described herein comprise 4 compound-containing units and optionally 3 compound-free units. In certain embodiments, the kits described herein comprise 5 compound-containing units and optionally 2 compound-free units. In certain embodiments, the kits described herein comprise 6 compound-containing units and optionally 1 compound-free unit.

In some embodiments, the kits described herein comprise 12 to 24 compound-containing units and optionally 4 to 16 compound-free units. In certain embodiments, the kits described herein comprise 12 compound-containing units and optionally 16 compound-free units. In certain embodiments, the kits described herein comprise 16 compound-containing units and optionally 12 compound-free units. In certain embodiments, the kits described herein comprise 20 compound-containing units and optionally 8 compound-free units. In certain embodiments, the kits described herein comprise 24 compound-containing units and optionally 4 compound-free units.

In some embodiments, each of the compound containing units in the kits described herein comprises 10-400 mg or 20-200 mg of Compound 1 or an equivalent dose of a pharmaceutically acceptable salt or deuterated form thereof, or a crystalline or polymorph form of Compound 1. In some embodiments, each of the compound-containing units comprises about 10 mg, about 50 mg, about 60 mg, about 120 mg, about 150 mg, about 180 mg, about 240 mg, about 300 mg, or about 400 mg of Compound 1 or an equivalent dose of a pharmaceutically acceptable salt or deuterated form thereof, or a crystalline or polymorph form of Compound 1. In certain embodiments, each of the compound-containing units comprises about 10 mg of Compound 1 or the pharmaceutically acceptable salt or deuterated form thereof, or a crystalline or polymorph form of Compound 1. In certain embodiments, each of the compound-containing units comprises about 50 mg of Compound 1 or an equivalent dose of the pharmaceutically acceptable salt or deuterated form thereof, or a crystalline or polymorph form of Compound 1. In certain embodiments, each of the compound-containing units comprises about 60 mg of Compound 1 or an equivalent dose of a pharmaceutically acceptable salt or deuterated form thereof, or a crystalline or polymorph form of Compound 1. In certain embodiments, each of the compound-containing units comprises about 120 mg of Compound 1 or an equivalent dose of pharmaceutically acceptable salt or deuterated form thereof, or a crystalline or polymorph form of Compound 1. In certain embodiments, each of the compound-containing units comprises about 150 mg of Compound 1 or an equivalent dose of pharmaceutically acceptable salt or deuterated form thereof, or a crystalline or polymorph form of Compound 1. In certain embodiments, each of the compound-containing units comprises about 180 mg of Compound 1 or the pharmaceutically acceptable salt or deuterated form thereof, or a crystalline or polymorph form of Compound 1. In certain embodiments, each of the compound-containing units comprises about 240 mg of Compound 1 or an equivalent dose of pharmaceutically acceptable salt or deuterated form thereof, or a crystalline or polymorph form of Compound 1. In certain embodiments, each of the compound-containing units comprises about 300 mg of Compound 1 or the pharmaceutically acceptable salt or deuterated form thereof, or a crystalline or polymorph form of Compound 1. In certain embodiments, each of the compound-containing units comprises about 400 mg of Compound 1 or an equivalent dose of pharmaceutically acceptable salt or deuterated form thereof, or a crystalline or polymorph form of Compound 1. Each of the compound containing units in the kits described herein may comprise about the same amount of Compound 1 or a pharmaceutically acceptable salt or deuterated form thereof, or a crystalline or polymorph form of Compound 1.

In some embodiments, the kits described herein may comprise one or more packaging units. One or more packaging units may include, without being limited to, 1 packaging unit, 2 packaging units, 3 packaging units, 4 packaging units, 5 packaging units and 6 packaging units or more.

Each packaging unit may comprise 12 to 24 compound-containing units and optionally 4 to 16 compound-free units. In certain embodiments, each packaging unit comprises 12 compound-containing units and optionally 16 compound-free units. In certain embodiments, each packaging unit comprises 16 compound-containing units and optionally 12 compound-free units. In certain embodiments, each packaging unit comprises 20 compound-containing units and optionally 8 compound-free units. In certain embodiments, each packaging unit comprises 24 compound-containing units and optionally 4 compound-free units.

In some embodiments, each compound-containing unit comprises Compound 1 or a pharmaceutically acceptable salt or deuterated form thereof, and oral administration of a compound-containing unit provides a pharmacokinetic profile for Compound 1 characterized by one or more of the following features:
(i) a mean Tₘₐₓ of at least about 2, 4, or 6 h; and/or
(ii) a mean Cₘₐₓ which is less than about 1,000 ng/ml, 5,000 ng/ml, or 10,000 ng/mL.

In some embodiments, the oral administration of the said daily active dosage unit provides a pharmacokinetic profile that may further be characterized by a mean AUC0 h-tlast of at least 3000 (h*ng/ml), more preferably of at least 3500 (h*ng/ml) when measured in rat administered Compound 1 or a pharmaceutically acceptable salt or deuterated form thereof at 50 mg/kg. In another specific embodiment, the mean Tₘₐₓ may range from about 2.2 hrs to about 6 hrs. In another embodiment, the mean Cₘₐₓ may range from about 15 ng/ml to about 30 ng/ml or 1,000-12,000 ng/ml in rat administered Compound 1 or a pharmaceutically acceptable salt or deuterated form thereof at 50 mg/kg.

In some embodiments of the kits described herein, the compound-free unit is a pharmaceutically acceptable placebo. In certain embodiments, the kits comprise 28 compound-containing units and zero units of a pharmaceutically acceptable placebo. In certain embodiments, the kits comprise 24 compound-containing units and 4 units of a pharmaceutically acceptable placebo. In certain embodiments, the kits comprise 20 compound-containing units and 8 units of a pharmaceutically acceptable placebo. In certain embodiments, the kits comprise 16 compound-containing units and 12 units of a pharmaceutically acceptable placebo. In certain embodiments, the kits comprise 12 compound-containing units and 16 units of a pharmaceutically acceptable placebo.

In some embodiments, the kits of the present disclosure comprise one or more packaging units, with each packing unit designed to provide daily compound-containing units, and optionally pharmaceutically acceptable placebo units, for four 7-day cycles. For each 7 day cycle, at least 3 compound-containing units, and optionally at least 1 pharmaceutically acceptable placebo unit, are provided, resulting in a kit comprising at least 12 compound-containing units and optionally at least 4 placebo units.

The packaging units as described above may be a conventional blister pack comprising the appropriate number of compound-containing units in a sealed blister pack with a cardboard, paperboard, foil or plastic backing and enclosed in a suitable cover. Each blister container may be conveniently numbered or marked in order to facilitate compliance. The packaging unit may contain daily compound-containing units in the order in which they are to be taken.

In embodiments, the disclosure includes kits comprising compound-containing units comprising Compound 2, or a pharmaceutically acceptable salt or deuterated form thereof, or a crystalline or polymorph form of Compound 2, and each of said compound-free unit does not comprise Compound 2 or the pharmaceutically acceptable salt or deuterated form thereof, or a crystalline or polymorph form of Compound 2, according to the above described kits or packaging.

The kits described herein may comprise other appropriate components such as instructions for use.

### NUMBERED EMBODIMENTS

1. A method of treating Duchenne Muscular Dystrophy (DMD) in a subject in need thereof, comprising administering a therapeutically effective amount of Compound 1, or a pharmaceutically acceptable salt or deuterated form thereof (including crystalline and polymorph forms) for a treatment period, wherein each treatment period comprises two or more consecutive treatment cycles,

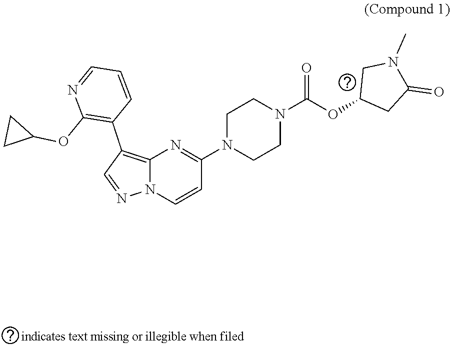 [SMILES: CN1C[C@@H](OC(=O)N2CCN(c3ccn4ncc(-c5cccnc5OC5CC5)c4n3)CC2)CC1=O]

wherein each treatment cycle comprises administering Compound 1 or the pharmaceutically acceptable salt or deuterated form thereof (including crystalline and polymorph forms) to the subject for 3, 4, 5, 6, 7, 8, 9, or 10 consecutive days, followed by an interval of 1, 2, 3, or 4 days wherein Compound 1 or the pharmaceutically acceptable salt or deuterated form thereof is not administered to the subject.

1a. A method of treating Duchenne Muscular Dystrophy (DMD) in a subject in need thereof, comprising administering a therapeutically effective amount of Compound 1 or a pharmaceutically acceptable salt thereof for a treatment period, wherein each treatment period comprises two or more consecutive treatment cycles,

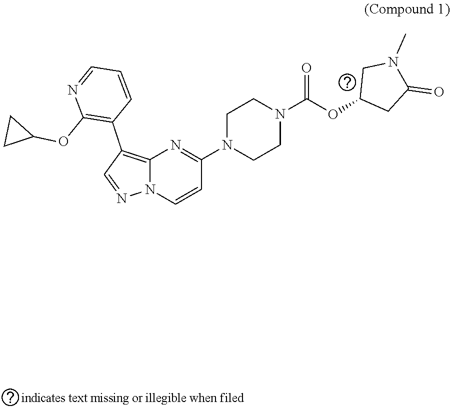 [SMILES: CN1C[C@@H](OC(=O)N2CCN(c3ccn4ncc(-c5cccnc5OC5CC5)c4n3)CC2)CC1=O]

wherein each treatment cycle comprises administering Compound 1 or the pharmaceutically acceptable salt or deuterated form thereof (including crystalline and polymorph forms) to the subject for 3, 4, 5, 6, 7, 8, 9, or 10 consecutive days, followed by an interval of 1, 2, 3, or 4 days wherein Compound 1 or the pharmaceutically acceptable salt or deuterated form thereof is not administered to the subject.

2. The method of embodiment 1 or 1a, wherein:
(a) Compound 1 or the pharmaceutically acceptable salt or deuterated form thereof (including crystalline and polymorph forms) is administered for at least 3 consecutive days, followed by an interval of at least 1 day wherein Compound 1 or the pharmaceutically acceptable salt or deuterated form thereof (including crystalline and polymorph forms) is not administered;
(b) Compound 1 or the pharmaceutically acceptable salt or deuterated form thereof (including crystalline and polymorph forms) is administered for at least 4 consecutive days, followed by an interval of at least 1 day wherein Compound 1 or the pharmaceutically acceptable salt or deuterated form thereof (including crystalline and polymorph forms) is not administered;
(c) Compound 1 or the pharmaceutically acceptable salt or deuterated form thereof (including crystalline and polymorph forms) is administered for at least 5 consecutive days, followed by an interval of at least 1 day wherein Compound 1 or the pharmaceutically acceptable salt or deuterated form thereof (including crystalline and polymorph forms) is not administered; or
(d) Compound 1 or the pharmaceutically acceptable salt or deuterated form thereof (including crystalline and polymorph forms) is administered for at least 6 consecutive days, followed by an interval of at least 1 day wherein Compound 1 or the pharmaceutically acceptable salt or deuterated form thereof (including crystalline and polymorph forms) is not administered.

3. The method of any one of embodiments 1, 1a, and 2, wherein the treatment period comprises at least one 7-day treatment cycle, wherein Compound 1 or the pharmaceutically acceptable salt or deuterated form thereof (including crystalline and polymorph forms) is administered for 3 or more days of each 7-day treatment cycle and not administered for 1 or more day of each 7-day treatment cycle.

4. The method of any one of embodiments 1-3, wherein each treatment cycle is the same or different.

5. The method of embodiment 3, wherein each treatment cycle is a 7-day treatment cycle wherein:
(a) Compound 1 or the pharmaceutically acceptable salt or deuterated form thereof (including crystalline and polymorph forms) is administered for 3 consecutive days, followed by an interval of 4 days wherein Compound 1 or the pharmaceutically acceptable salt or deuterated form thereof (including crystalline and polymorph forms) is not administered;
(b) Compound 1 or the pharmaceutically acceptable salt or deuterated form thereof (including crystalline and polymorph forms) is administered for 4 consecutive days, followed by an interval of 3 days wherein Compound 1 or the pharmaceutically acceptable salt or deuterated form thereof (including crystalline and polymorph forms) is not administered;
(c) Compound 1 or the pharmaceutically acceptable salt or deuterated form thereof (including crystalline and polymorph forms) is administered for 5 consecutive days, followed by an interval of 2 days wherein Compound 1 or the pharmaceutically acceptable salt or deuterated form thereof (including crystalline and polymorph forms) is not administered; or
(d) Compound 1 or the pharmaceutically acceptable salt or deuterated form thereof (including crystalline and polymorph forms) is administered for 6 consecutive days, followed by an interval of 1 day wherein Compound 1 or the pharmaceutically acceptable salt or deuterated form thereof (including crystalline and polymorph forms) is not administered.

6. The method of embodiment 5, wherein for each 7-day treatment cycle, Compound 1 or the pharmaceutically acceptable salt or deuterated form thereof (including crystalline and polymorph forms) is administered for 5 consecutive days, followed by an interval of 2 days wherein Compound 1 or the pharmaceutically acceptable salt or deuterated form thereof (including crystalline and polymorph forms) is not administered.

7. The method of any one of embodiments 1-6, wherein Compound 1 or the pharmaceutically acceptable salt or deuterated form thereof (including crystalline and polymorph forms) is administered once daily.

8. The method of any one of embodiments 1-7, wherein 10-400 mg of Compound 1 or the pharmaceutically acceptable salt or deuterated form thereof is administered to the subject daily.

9. The method of embodiment 8, wherein about 10 mg of Compound 1 or the pharmaceutically acceptable salt or deuterated form thereof (including crystalline and polymorph forms) is administered to the subject daily.

10. The method of embodiment 8, wherein the compound-containing unit comprises about 50 mg of Compound 1, or a pharmaceutically acceptable salt or deuterated form thereof (including crystalline and polymorph forms) is administered to the subject daily.

11. The method of embodiment 8, wherein about 60 mg of Compound 1 or the pharmaceutically acceptable salt or deuterated form thereof (including crystalline and polymorph forms) is administered to the subject daily.

12. The method of embodiment 8, wherein about 120 mg of Compound 1 or the pharmaceutically acceptable salt or deuterated form thereof (including crystalline and polymorph forms) is administered to the subject daily.

13. The method of embodiment 8, wherein about 150 mg of Compound 1 or the pharmaceutically acceptable salt or deuterated form thereof (including crystalline and polymorph forms) is administered to the subject daily.

14. The method of embodiment 8, wherein about 180 mg of Compound 1 or the pharmaceutically acceptable salt or deuterated form thereof (including crystalline and polymorph forms) is administered to the subject daily.

15. The method of embodiment 8, wherein about 240 mg of Compound 1 or the pharmaceutically acceptable salt or deuterated form thereof (including crystalline and polymorph forms) is administered to the subject daily.

16. The method of embodiment 8, wherein about 300 mg of Compound 1 or the pharmaceutically acceptable salt or deuterated form thereof (including crystalline and polymorph forms) is administered to the subject daily.

17. The method of embodiment 8, wherein about 400 mg of Compound 1 or the pharmaceutically acceptable salt or deuterated form thereof (including crystalline and polymorph forms) is administered to the subject daily.

18. The method of any one of embodiments 1-17, wherein Compound 1 or the pharmaceutically acceptable salt or deuterated form thereof (including crystalline and polymorph forms) is administered orally.

19. The method of any one of embodiments 1-18, wherein force generation by muscles is increased, forced vital capacity (FVC) is increased, and/or 99th Percentile Maximum Effort is increased in the subject after administration of Compound 1 or the pharmaceutically acceptable salt or deuterated form thereof (including crystalline and polymorph forms).

20. The method of embodiment 19, wherein the force generation by muscles is measured by upper extremity dynamometry.

21. The method of embodiment 19, wherein the FVC is measured by spirometry.

22. The method of embodiment 19, wherein the 99th Percentile Maximum Effort is measured by a wearable monitoring device.

23. The method of any one of embodiments 1-22, wherein the blood plasma level of Compound 1 or the pharmaceutically acceptable salt or deuterated form thereof (including crystalline and polymorph forms) in the subject is:
(a) above the IC50 for a period of time within the 24 hours following each administration of the Compound 1 or the pharmaceutically acceptable salt or deuterated form thereof (including crystalline and polymorph forms), and wherein the blood plasma level is below the IC50 during the interval wherein Compound 1 or the pharmaceutically acceptable salt or deuterated form thereof (including crystalline and polymorph forms) is not administered; and/or
(b) above about 130 ng/ml for a period of time within the 24 hours following each administration of Compound 1 or the pharmaceutically acceptable salt or deuterated form thereof (including crystalline and polymorph forms) is administered, and wherein the blood plasma level is below about 130 ng/ml during the interval wherein Compound 1 or the pharmaceutically acceptable salt or deuterated form thereof (including crystalline and polymorph forms) is not administered.

24. The method of embodiment 23, wherein the blood plasma level of Compound 1 or the pharmaceutically acceptable salt or deuterated form thereof (including crystalline and polymorph forms) is above the IC50 or above about 130 ng/ml for at least about 6 hours during the 24 hours following each administration of the Compound 1 or the pharmaceutically acceptable salt or deuterated form thereof (including crystalline and polymorph forms).

25. The method of embodiment 24, wherein the blood plasma level is above the IC50 for at least about 6-8 hours during the during the 24 hours following each administration of the Compound 1 or the pharmaceutically acceptable salt or deuterated form thereof (including crystalline and polymorph forms).

26. The method of any one of embodiments 1-25, wherein the subject in need thereof is ambulatory.

27. The method of any one of embodiments 1-25, wherein the subject in need thereof is non-ambulatory.

28. A kit for treatment of Duchenne muscular dystrophy (DMD), comprising at least 3 compound-containing units and optionally at least 1 compound-free unit, wherein each of said compound-containing units comprises Compound 1 or a pharmaceutically acceptable salt or deuterated form thereof (including crystalline and polymorph forms), and each of said compound-free unit does not comprise Compound 1 or the pharmaceutically acceptable salt or deuterated form thereof (including crystalline and polymorph forms).

29. The kit of embodiment 28, comprising:
(a) 3 compound-containing units and optionally 4 compound-free units;
(b) 4 compound-containing units and optionally 3 compound-free units;
(c) 5 compound-containing units and optionally 2 compound-free units; or
(d) 6 compound-containing units and optionally 1 compound-free units.

30. The kit of embodiment 28 or embodiment 29, comprising 12 to 24 compound-containing units and optionally 4 to 16 compound-free units.

31. The kit of embodiment 30, comprising:
(a) 12 compound-containing units and optionally 16 compound-free units;
(b) 16 compound-containing units and optionally 12 compound-free units;
(c) 20 compound-containing units and optionally 8 compound-free units; or
(d) 24 compound-containing units and optionally 4 compound-free units.

32. The kit of any one of embodiments 28-31, wherein each compound-containing unit comprises 10-400 mg of Compound 1 or the pharmaceutically acceptable salt or deuterated form thereof (including crystalline and polymorph forms).

33. The kit of embodiment 32, wherein each of the compound-containing units comprises about 10 mg, about 50 mg, about 60 mg, about 120 mg, about 150 mg, about 180 mg, about 240 mg, about 300 mg, or about 400 mg of Compound 1 or the pharmaceutically acceptable salt or deuterated form thereof (including crystalline and polymorph forms).

34. The kit of any one of embodiments 28-33, wherein each of the compound-containing units comprises the same amount of Compound 1 or the pharmaceutically acceptable salt or deuterated form thereof (including crystalline and polymorph forms).

35. A method of increasing the force generation by the skeletal muscles including diaphragm of a subject having Duchenne Muscular Dystrophy (DMD), comprising administering a therapeutically effective amount of Compound 1, or a pharmaceutically acceptable salt or deuterated form thereof (including crystalline and polymorph forms), for a treatment period, wherein each treatment period comprises two or more consecutive treatment cycles,
wherein each treatment cycle comprises administering Compound 1 or the pharmaceutically acceptable salt or deuterated form thereof (including crystalline and polymorph forms) to the subject for 3, 4, 5, 6, 7, 8, 9, or 10 consecutive days, followed by an interval of 1, 2, 3, or 4 days wherein Compound 1 or the pharmaceutically acceptable salt or deuterated form thereof (including crystalline and polymorph forms) is not administered to the subject.

36. The method of embodiment 35, wherein each treatment cycle comprises administering Compound 1 or the pharmaceutically acceptable salt or deuterated form thereof (including crystalline and polymorph forms) to the subject for 5 consecutive days, followed by an interval of 2 days wherein Compound 1 or the pharmaceutically acceptable salt or deuterated form thereof (including crystalline and polymorph forms) is not administered to the subject.

37. The method of embodiment 36, wherein about 60 mg or about 120 mg of Compound 1 or the pharmaceutically acceptable salt or deuterated form thereof (including crystalline and polymorph forms) are orally administered to the subject daily for the 5 consecutive days.

38. The method of any one of embodiments 1-27 and 35-37, wherein the average grip strength of the subject increased about 2-fold, about 3-fold, about 4-fold, or about 5-fold after a treatment period.

39. The method of embodiment 38, wherein the treatment period is for 28 days.

### EXAMPLES

### Example 1: Compound 1 and Compound 2 Induce Asymmetric Division

Ex vivo fiber assays were conducted to determine the effect of Compound 1 and Compound 2 on MyoG+ cells and % MyoG. The number of Pax7+ cells was also measured. Briefly, the ex vivo fiber assays were conducted as follows:

### Media Preparation

(a) Base Media: DMEM (1×)+1 g/L D-Glucose+L-Glutamine+110 mg/L Sodium Pyruvate (Gibco 11885-084)+2% penstrep.
(b) Digestion Media: 700 u/ml Collagenase Type I in Base Media. (at least 2 ml aliquots per mouse in 15 ml tube).
(c) Wash Media (AKA Titeration media, Isolation media): Base Media+20% FBS.
(d) Growth media: Wash media 198 mL+1% CEE (2 ml)+2.5 ul FGF makes 100 ml. Note: All the media prepared were filtered through 0.2 μM syringe.

### Dissection and FDB Muscle Harvest

(e) The FBD muscle is the most superficial muscle on the palm of the foot.
(f) FDB muscle harvested using sterilize Surgical Tools.
(g) First, skin of the hand feet is de-gloved, peeled towards the toes leaving the skin attached at the end of the foot.
(h) All the five tendons attached to the FDB to the toes are cut and pull the FDB muscle is pulled away from the foot towards the heel, cutting the connective tissue with a scalpel or scissors at a 45-degree angle.
(i) Immediately FDB muscle is placed in a 6-well plate with 5 mL media added, digested with collagenase I solution for 3.0 hrs+/−15 mins in the incubator with gentle swirl every 20-30 mins.

### FDB Single Fiber Separation Post-Digestion

(j) After incubation, digestive media with muscle is poured into 60 mm Dish.
(k) With a flame-polished wide-bore glass pipette media is titrated next to the muscle until fibers come loose from the tendon.
(l) Single fibers released into the media are washed 2× with warm fiber wash media.
(m) In the final wash, 3 mL of wash media was added.
(n) Pre-plating was done for 2 hrs+/−30 mins to allow adherence of fibroblasts.
(o) Single FDB fibers were distributed in a horse serum coated 12-well plate.

### Addition of Drug

(p) Required amount of drug is thawed on ice and drug dilutions were prepared.
(q) Drug conditions used:
   Compound 1 freebase Batch: 7. Mol wt: 477.53
   DMSO control, 30 nM, 100 nM, 300 nM and 1000 nM (1 μM) for both Compound 1 and Compound 2
   DMSO control, 30 nM, 100 nM, 300 nM and 1000 nM (1 μM) for Compound 1 and 300 nM for Compound 2.
(r) Growth media was aspirated from each well in the 12 well plates (one at a time).
(s) 2 mL of Growth media with drug at the final concentrations prepared was added to each well.
(t) Plate was incubated for 72 hrs at 37° C.

### Immunostaining

(u) Single FDB fibers were fixed with 4% PFA for 10 mins, permeabilized with 0.1% Triton X-100. 0.1M Glycine for 45 mins, followed by 3×PBS washes.
(v) Primary antibody is diluted in 2×AES buffer (20 mM glycine+0.2% Triton-X+0.1% Tween20+0.2% H₂O₂ in 2×PBS).
(w) In 20 mL 2×AES buffer, 20 mL of Pax7 hybridoma (1:1) and 53 μL of rabbit anti-Myogenin ab124800 (1:750) was added. The primary Ab solution was added to Add 1 mL to each well and incubated at 4 degrees C. O/N. Following O/N incubation MHC IgG2b hybridoma was added to each well ate a ratio of 3:1 (3 parts MHC: 1 part PBS) and incubated at RT for 1 hour. After incubation, 3× washes with PBS was performed.
(x) Secondary antibodies goat anti-msIgG1 Alexa546 Cat. No A21123, goat anti-rabbit A488, goat anti-msIgG2b A647 were diluted at 1:1500, filtered through PES filter (low binding filter).
(y) Secondary antibody solution was added to each well and incubated 1 h at room temperature (keep in the dark), washed at least 2 times in PBS, followed by DAPI (1:10,000 in PBS).
(z) Fibers were distributed into 96-well plate carefully after 5-10 mins incubation with DAPI.
(aa) Plate was run on the OHRI Operetta for scanning and analysis.

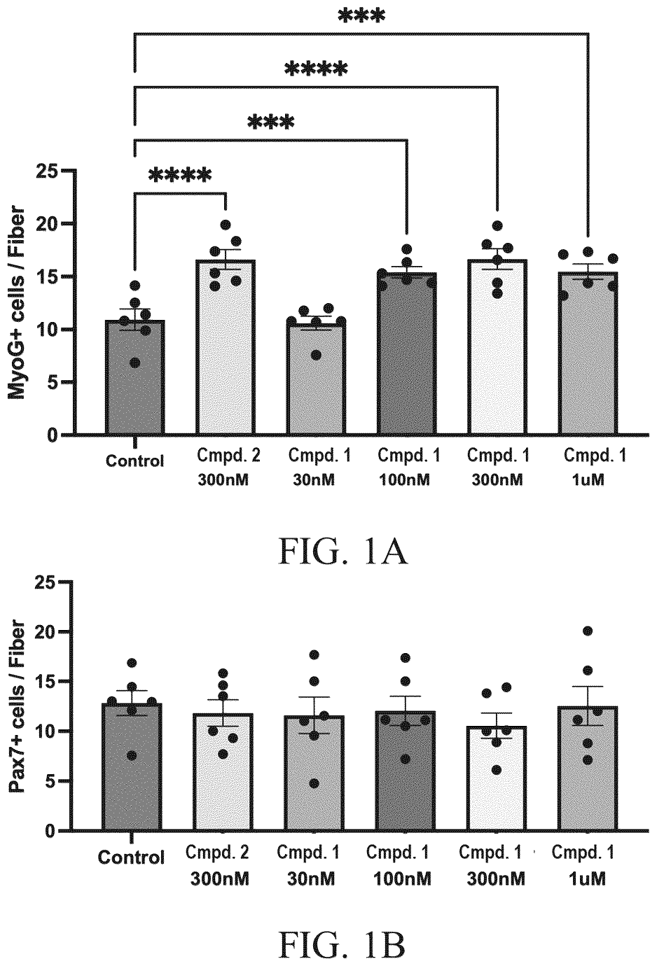

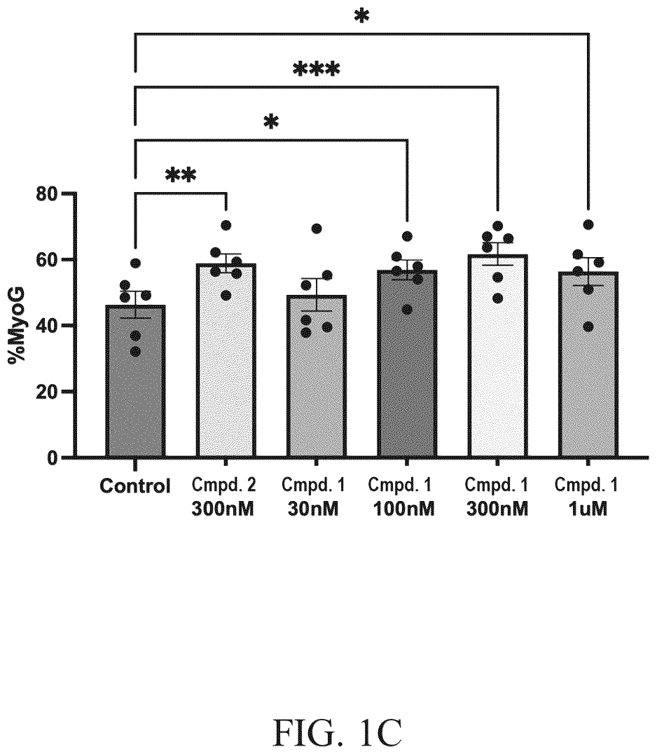

As shown in FIGS. 1A and 1C, there was a significant increase in the number of MyoG+ cells per fiber and % MyoG with after treatment with both Compound 2 and Compound 1. There was no significant difference observed in the number of Pax7+ cells, as shown in FIG. 1B.

### Example 2: Pharmacokinetics of Compound 1 and Compound 2

Studies were conducted to determine the pharmacokinetics of various salt forms of Compound 1 and Compound 2 in male C57BL/6J mice, Sprague-Dawley Rats, and Beagle dogs. Animals were administered Compound 1, Compound 2, or Vehicle either intraperitoneally (IP), per os (PO), or intravenously (IV). Doses tested included 1 mg/kg, 3 mg/kg, 10 mg/kg, and/or 30 mg/kg.

The pharmacokinetic parameters assessed include: Cₘₐₓ (observed peak or maximum concentration); Tₘₐₓ (observed time of peak concentration); T_{1/2} (terminal half-life); Vz (volume of distribution based on terminal phase); AUC_{inf} (area under the concentration-time curve computed from time zero to infinity); AUCₗₐₛₜ (area under the concentration-time curve computed from time zero to the time of the last quantifiable concentration); and F (bioavailability).

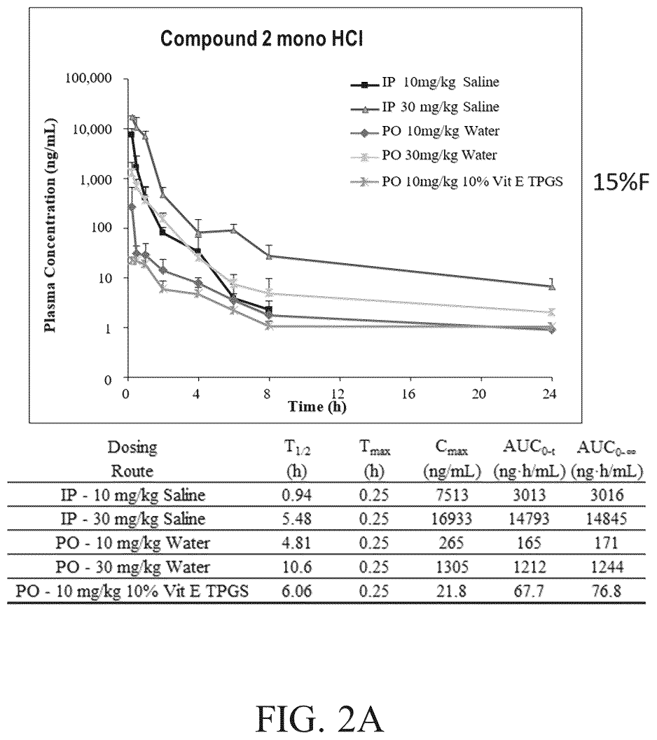

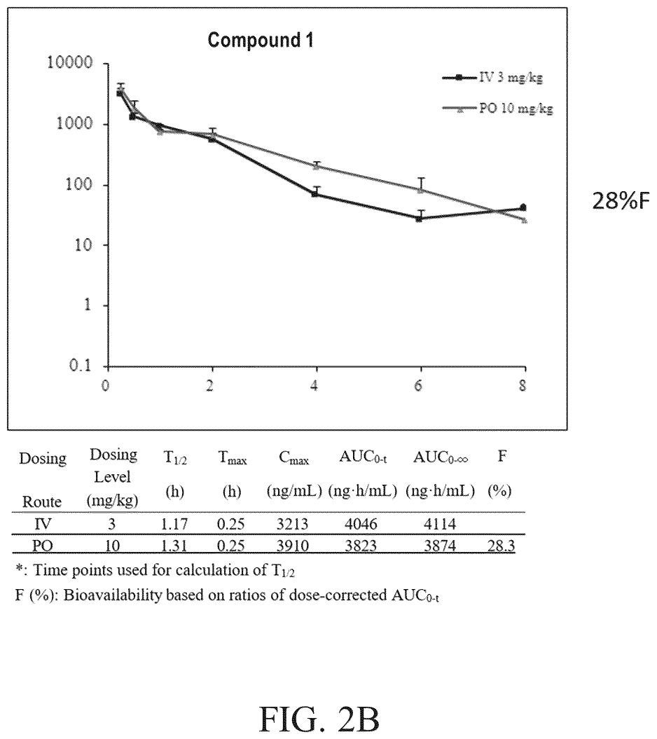

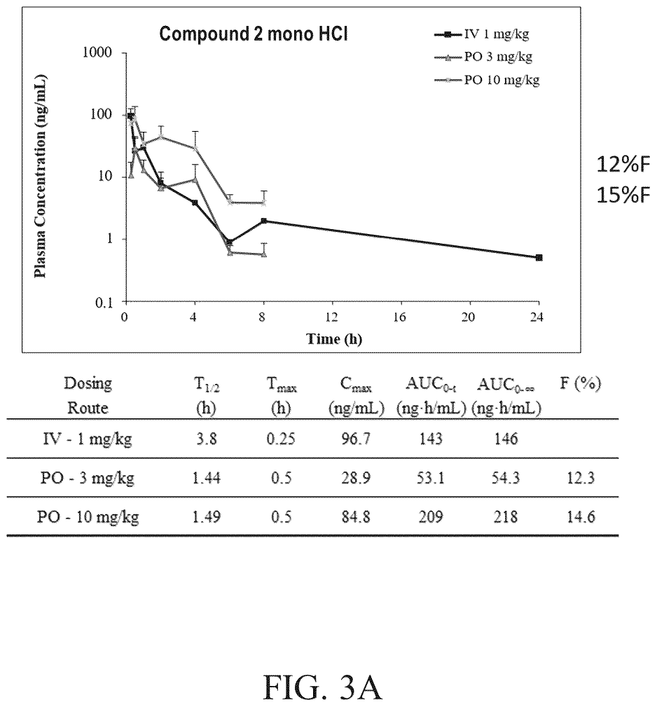

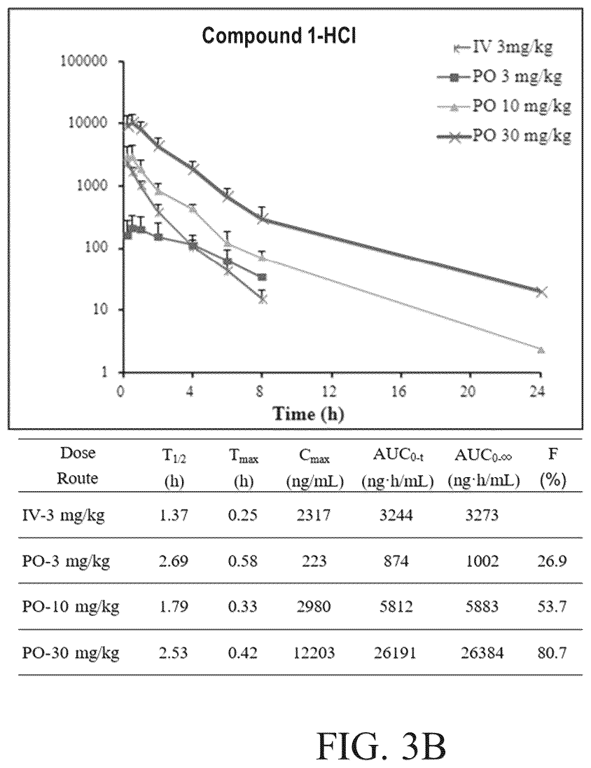

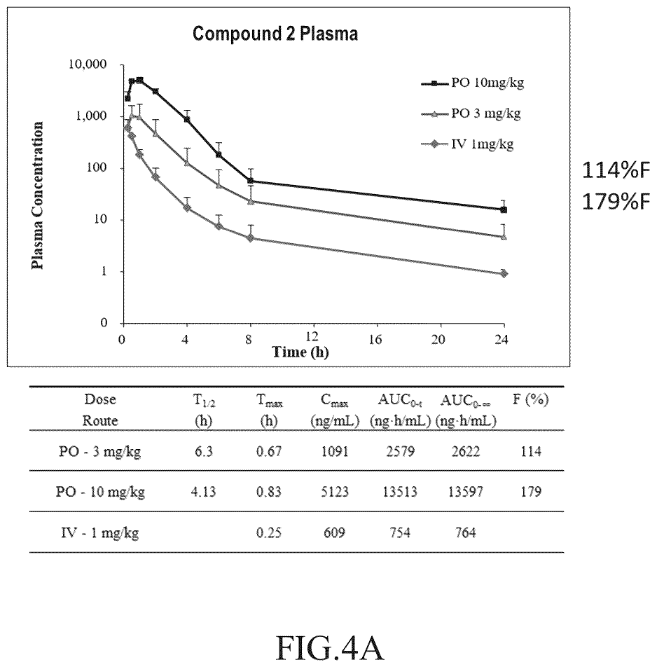

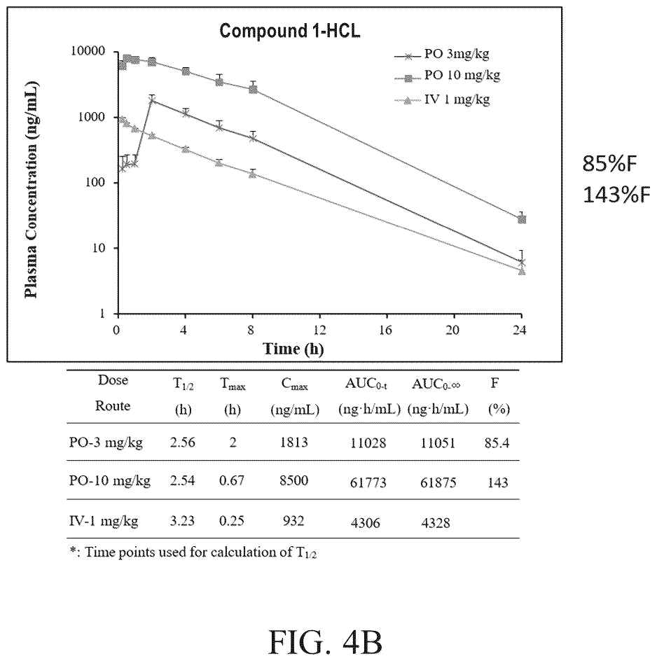

PK parameters calculated from the mouse study are presented in FIGS. 2A and 2B. PK parameters calculated from the rat study are presented in FIGS. 3A and 3B. PK parameters calculated from the beagle study are presented in FIGS. 4A and 4B.

### Example 3: Pharmacokinetics of Compound 1 in Rats

An in vivo pharmacokinetic (PK) study of Compound 1 was conducted in male Sprague Dawley rats to determine the concentration of Compound 1 in plasma following a single intravenous (IV) or oral (PO) dose administration of Compound 1. PK parameters including bioavailability were calculated.

Male Sprague Dawley rats (9 weeks of age) were received from Charles River Laboratories and allowed to acclimate for at least 2 days prior to study. The study involved administering Compound 1 to male Sprague Dawley rats, resulting in 8 groups for 3 test articles. For Compound 1 oxalate salt, an intravenous dose of 3 mg/kg was given, prepared as a dosing solution with a target concentration of 3 mg/mL in a volume of 1 mL/kg. Oral doses for the Compound 1 oxalate salt and Compound 1 HCl salt were both administered at a dose of 10, 50, and 150 mg/kg, individually prepared as dosing solutions with target concentrations of 2, 10, and 30 mg/mL, respectively, in a volume of 5 mL/kg. For Compound 1 phosphate salt, an oral dose of 10 mg/kg was given, prepared as a dosing solution with a target concentration of 2 mg/mL in a volume of 5 mL/kg.

Blood was collected and processed to plasma for analysis. Using a fit for purpose liquid chromatography/mass spectrometry (LC-MS/MS) method, concentrations of Compound 1 were quantified. The pharmacokinetic parameters were calculated using the concentration data generated from this analysis.

Throughout the study, no mortality, morbidity, or adverse clinical observations were observed in the animal models, indicating good tolerance to the administered doses of the test articles.

### Test Articles

The 3 test articles, Compound 1 oxalate salt, Compound 1 HCl salt, and Compound 1 phosphate salt, were stored at 22±5° C.

### Study Design

Table 4 below provides a summary of the different groups in the study, including the number of animals in each group, the compound ID, vehicle, dose level (in mg/kg), dose concentration (in mg/mL), dose volume (in mL/kg), and dose route.

TABLE 4
Sprague Dawley Rats Study design

| | | | | Dose | | |
|---|---|---|---|---|---|---|
| | Number | | Dose | Concen- | Dose | |
| | of | | Level | tration | Volume | Dose |
| Group | Animals | Compound | (mg/kg) | (mg/mL) | (mL/kg) | Route |
| 1 | 3 | Compound 1 | 3 | 3 | I | IV |
| | | oxalate salt | | | | |
| 2 | 3 | Compound 1 | 10 | 2 | 5 | PO |
| | | oxalate salt | | | | |
| 3 | 3 | Compound 1 | 50 | 10 | 5 | PO |
| | | oxalate salt | | | | |
| 4 | 3 | Compound 1 | 150 | 30 | 5 | PO |
| | | oxalate salt | | | | |
| 5 | 3 | Compound 1 | 10 | 2 | 5 | PO |
| | | HCI salt | | | | |
| 6 | 3 | Compound 1 | 50 | 10 | 5 | PO |
| | | HCI salt | | | | |
| 7 | 3 | Compound 1 | 150 | 30 | 5 | PO |
| | | HCI salt | | | | |
| 8 | 3 | Compound 1 | 10 | 2 | 5 | PO |
| | | phosphate | | | | |
| | | salt | | | | |

### Dose Procedures

Intravenous formulations were administered via jugular vein cannula. Following intravenous dose administration, a flush of ~0.5 mL of 0.9% N Saline was administered. Dosing catheters were tied off to prevent re-access. Doses were calculated based on pretreatment body weights (kg) and a dose volume of 1 mL/kg.

Oral formulations were administered by oral gavage using a ball tipped gavage needle. Doses were calculated based on pretreatment body weights (kg) and a dose volume of 5 mL/kg.

### Blood Sample Collection and Processing

Following dose administration for n=3 per group, serial blood samples (approximately 250 μL) were collected via jugular vein cannula into tube containing K₂EDTA. Samples were stored on wet ice until processed for plasma via centrifugation (3500 rpm for 10 minutes at 5° C.) within 30 minutes of collection. Plasma was transferred into matrix tubes and then stored in an −80° C. freezer until samples were transferred for analysis. Blood samples were collected 0.25, 0.5, 1, 2, 4, 6, 8, and 24 hours post dose.

### Plasma Sample Processing and Analysis

Plasma samples were analyzed for concentrations of Compound 1. A stock solution of Compound 1 was prepared at 0.913 mg/mL in DMSO for use as a standard. To a clean 96-well plate, 10 μL of plasma samples, calibration standards, QCs and blanks were aliquoted into appropriate wells. 100 μL of an internal standard cocktail solution in acetonitrile was added to all wells except for double blanks; double blanks received 100 μL of acetonitrile. The plate was then covered, vortexed and centrifuged at 4000 rpm for 10 minutes. 50 μL of the supernatant was then transferred to a clean 96-well plate containing 50 μL of water.

Compound 1 concentrations in plasma following intravenous and oral administration to male Sprague Dawley rats at targeted dose levels of 3 mg/kg (IV), 10 mg/kg (PO), 50 mg/kg (PO), or 150 mg/kg (PO) were measured and the results for each group are presented in Table 5 below.

TABLE 5
Compound 1 Concentrations (ng/mL) in Rat Plasma

| | Compound 1 Concentrations (ng/ml) in Rat Plasma | | | | | | | | | | | | | | | |
|---|---|---|---|---|---|---|---|---|---|---|---|---|---|---|---|---|
| | Group 1 | | Group 2 | | Group 3 | | Group4 | | Group 5 | | Group 6 | | Group 7 | | Group 8 | |
| Time | IV | | PO | | PO | | PO | | PO | | PO | | PO | | PO | |
| Point | (3 mg/kg) | | (10 mg/kg) | | (50 mg/kg) | | (150 mg/kg) | | (10 mg/kg) | | (50 mg/kg) | | (150 mg/kg) | | (10 mg/kg) | |
| (hr) | Mean | SD | Mean | SD | Mean | SD | Mean | SD | Mean | SD | Mean | SD | Mean | SD | Mean | SD |
| 0.250 | 2310 | 375 | 356 | 144 | 1190 | 191 | 1867 | 1348 | 214 | 109 | 6303 | 2022 | 9050 | 771 | 218 | 45.6 |
| 0.500 | 1713 | 323 | 814 | 265 | 3240 | 863 | 5653 | 2047 | 387 | 206 | 7273 | 3141 | 10350 | 1429 | 441 | 164 |
| 1.00 | 1214 | 353 | 1094 | 268 | 5177 | 1813 | 9120 | 1385 | 808 | 228 | 7363 | 2081 | 12000 | 1637 | 839 | 372 |
| 2.00 | 573 | 250 | 1034 | 270 | 4977 | 1782 | 8970 | 1965 | 732 | 307 | 5200 | 1255 | 10967 | 907 | 795 | 128 |
| 4.00 | 242 | 123 | 1078 | 29 | 5027 | 1260 | 9907 | 2870 | 526 | 170 | 4443 | 287 | 10330 | 1431 | 659 | 213 |
| 6.00 | 127 | 70.6 | 728 | 314 | 4280 | 1027 | 7373 | 2421 | 441 | 89.5 | 4477 | 731 | 10800 | 1645 | 409 | 195 |
| 8.00 | 74.5 | 42.5 | 480 | 181 | 3050 | 1414 | 7630 | 3472 | 245 | 22.9 | 3803 | 820 | 15033 | 4325 | 269 | 92.8 |
| 24.0 | 7.75 | 3.68 | 20.0 | 4.96 | 134 | 129 | 3633 | 2452 | 42.2 | 51.9 | 318 | 500 | 2550 | 1064 | 27.3 | 23.6 |

Following intravenous administration of Compound 1 oxalate salt (Group 1), plasma concentrations showed moderate inter-animal variability.

Following oral administration of Compound 1 oxalate salt at 10 mg/kg (Group 2), mean plasma concentrations peaked at one hour post dose with mild inter-animal variability. Until two hours post dose, the mean plasma concentrations of Compound 1 were higher following intravenous dosing (3 mg/kg) than oral dosing (10 mg/kg).

Following oral administration of Compound 1 oxalate salt at 50 mg/kg (Group 3), mean plasma concentrations peaked at one hour post dose with moderate inter-animal variability.

Following oral administration of Compound 1 oxalate salt at 150 mg/kg (Group 4), mean plasma concentrations peaked at four hours post dose with moderate inter-animal variability.

Following oral administration of Compound 1 HCl salt at 10 mg/kg (Group 5), mean plasma concentrations peaked at one hour post dose with moderate inter-animal variability.

Following oral administration of Compound 1 HCl salt at 50 mg/kg (Group 6), mean plasma concentrations peaked at one hour post dose with mild inter-animal variability.

Following oral administration of Compound 1 HCl salt at 150 mg/kg (Group 7), mean plasma concentrations peaked at eight hours post dose with mild inter-animal variability.

Following oral administration of Compound 1 phosphate salt at 10 mg/kg (Group 8), mean plasma concentrations peaked at one hour post dose with mild inter-animal variability.

### Pharmacokinetic Parameter Estimates

Pharmacokinetic parameter estimates were calculated from individual animal Compound 1 plasma concentration-time data using the actual nominal dose, sampling times, and noncompartmental methods. The concentration-time data were analyzed to fit either intravenous-bolus plasma analysis model or extravascular dosing plasma analysis model using the software Phoenix WinNonLin 8.3.5.340 (Certara). The single-dose pharmacokinetic parameters assessed include, as appropriate: Cₘₐₓ (observed peak or maximum concentration); Tₘₐₓ (observed time of peak concentration); T_{1/2} (terminal half-life); Vz (volume of distribution based on terminal phase); AUC_{inf} (area under the concentration-time curve computed from time zero to infinity); AUCₗₐₛₜ (area under the concentration-time curve computed from time zero to the time of the last quantifiable concentration); C₀ (back-extrapolated concentration at time zero); CL (total body clearance); MRT (mean resistance time); and F (bioavailability).

Areas-under-the-plasma concentration-time curves (AUC) were estimated using linear-log trapezoidal rule. The area through the time of the last observable concentration is reported as AUCₗₐₛₜ−AUC extrapolated to infinity, AUC_{inf} was estimated by adding AUCₗₐₛₜ and the ratio of Cₗₐₛₜ/λz, where λz is the terminal phase constant. Apparent terminal half-life (T_{1/2}) was calculated as ln(2)/λz and determined using the slope of the log-linear terminal phase of the concentration-time curve, defined by a minimum of three plasma concentration-time points. Half-lives are reported if the correlation for the regression line, as measured by r squared, is >0.9 when rounded. After IV administration, volume of distribution (Vz) was calculated as Dose/λz*AUC_{inf}, clearance (Cl) was calculated as Dose/AUC_{inf}. Mean residence time (MRT) from the time of dosing to the time of last measurable concentration was calculated as AUMCₗₐₛₜ/AUCₗₐₛₜ. AUMC means area under the moment curve. For the extravascular model, the bioavailability (fraction of total dose that reaches the systemic circulation) was calculated as follows: (Average AUC_{last-PO}/Average AUC_{last-IV})*[Dose IV/Dose PO]*100.

Pharmacokinetic parameter estimates for Compound 1 in plasma following intravenous or oral administration of Compound 1 oxalate salt, Compound 1 chloride salt, or Compound 1 phosphate salt to male Sprague Dawley rats at target dose levels of 3 (IV), 10 (PO), 50 (PO), or 150 (PO) mg/kg are summarized in Table 6.

TABLE 6
PK parameter estimates for Compound 1 in plasma (mean/standard deviation)

| Group | 1 | 2 | 3 | 4 | 5 | 6 | 7 | 8 |
|---|---|---|---|---|---|---|---|---|
| Tₘₐₓ (h) | 0.250/ | 3.00/ | 3.00/ | 2.33/ | 1.33/ | .833/ | 7.33/ | 2.33/ |
| | 0.00 | 1.73 | 2.65 | 1.53 | 0.577 | 0.289 | 1.15 | 1.53 |
| Cₘₐₓ | 3124/ | 1287/ | 6157/ | 11533/ | 825/ | 7743/ | 15233/ | 910/ |
| (ng/ml) | 512 | 163 | 715 | 1332 | 217 | 2738 | 4131 | 305 |
| T_{1/2}(h) | 4.92/ | 3.53/ | 3.04/ | 9.40/ | 5.78/ | 3.91/ | 9.13 | 4.83/ |
| | 1.07 | 0.352 | NA | NA | 3.86 | 2.67 | NA | 1.82 |
| Extrapolated | 1.2/ | 0.9/ | 1.1/ | 16.6/ | 6.90/ | 3.4/ | 12.1/ | 3.1/ |
| (%) | 0.531 | 0.292 | 1.07 | NA | 9.93 | 5.65 | 7.84 | 3.00 |
| Number of | 3 per | 3 per | 3 per | 2 animals | 3 per | 3 per | 3 per | 3 per |
| regression | animal | animal | animal | had 3; 1 | animal | animal | animal | animal |
| points (n) per | | | | animal | | | | |
| animal | | | | had 0 | | | | |
| AUC₍₀₋ₓ₎ | 4851/ | 10857/ | 59994/ | 155178/ | 6379/ | 72236/ | 229554/ | 6821/ |
| (h*ng/ml) | 1601 | 2365 | 10014 | 30482 | 1392 | 8058 | 27694 | 1989 |
| AUC_{(0-INF)} | 4905/ | 10959/ | 55081/ | 207066/ | 6923/ | 75303/ | 255386/ | 7051/ |
| (h*ng/ml) | 1607 | 2374 | NA | NA | 1733 | 13120 | NA | 2092 |
| CL₍₀₋ₓ₎ | 671/ | NA | NA | NA | NA | NA | NA | NA |
| (mL/hr/kg) | 271 | | | | | | | |
| MRT₍₀₋ₓ₎ (h) | 2.66/ | NA | NA | NA | NA | NA | NA | NA |
| | 0.569 | | | | | | | |
| Vz (mL/kg) | 5021/ | NA | NA | NA | NA | NA | NA | NA |
| | 3132 | | | | | | | |
| F₍₀₋ₓ₎ (%) | NA | 67.1/ | 74.2/ | 64.0/ | NA | NA | NA | NA |
| | | 14.6 | 12.4 | 12.5 | | | | |
| F_{(0-INF)} (%) | NA | 67.0/ | 67.4/ | 84.4/ | NA | NA | NA | NA |
| | | 14.5 | NA | NA | | | | |

All values in Table 6 are presented as Mean/Standard deviation with n=3 unless otherwise noted.

Amongst the 10 mg/kg oral doses, the mean Cₘₐₓ was noted to be highest for Compound 1 oxalate salt at 1287 ng/mL, followed by Compound 1 phosphate salt at 910 ng/ml and Compound 1 HCl salt at 825 ng/mL. Amongst the 50 mg/kg oral doses, the mean Cₘₐₓ was noted to be highest for Compound 1 HCl salt at 7743 ng/mL, followed by Compound 1 oxalate salt at 6157 ng/mL. Amongst the 150 mg/kg oral doses, the mean Cₘₐₓ was noted to be highest for Compound 1 HCl salt at 15233 ng/mL, followed by Compound 1 oxalate salt at 11533 ng/ml.

Amongst the 10 mg/kg oral doses, the mean Tₘₐₓ was noted to be earliest for Compound 1 HCl salt at 1.33 hours, followed by Compound 1 phosphate salt at 2.33 hours and Compound 1 oxalate salt at 3.00 hours. Amongst the 50 mg/kg oral doses, the mean Tₘₐₓ was noted to be earliest for Compound 1 HCl salt at 0.83 hours, followed by Compound 1 oxalate salt at 3.00 hours. Amongst the 150 mg/kg oral doses, the mean Tₘₐₓ was noted to be earliest for Compound 1 oxalate salt at 2.33 hours, followed by Compound 1 HCl salt at 7.33 hours.

For Compound 1 oxalate salt, the mean AUC₍₀₋ₓ₎ increased from 4851 hour*ng/mL at 3 mg/kg intravenously to 10857 hour*ng/mL at 10 mg/kg orally. Amongst the 10 mg/kg oral doses, mean AUC₍₀₋ₓ₎ was noted to be highest for Compound 1 oxalate salt at 10857 hour*ng/ml, followed by Compound 1 phosphate salt at 6821 hour*ng/mL and Compound 1 HCl salt at 6379 hour*ng/mL. Amongst the 50 mg/kg oral doses, mean AUC₍₀₋ₓ₎ was noted to be highest for Compound 1 HCl salt at 72236 h*ng/mL, followed by Compound 1 oxalate salt at 59994 h*ng/mL. Amongst the 150 mg/kg oral doses, mean AUC₍₀₋ₓ₎ was noted to be highest for Compound 1 HCl salt at 229554 h*ng/mL, followed by Compound 1 oxalate salt at 155178 h*ng/mL.

Amongst the Compound 1 oxalate salt oral doses, the mean F₍₀₋ₓ₎ was noted to be highest for 50 mg/kg at 74.2%, followed by 10 mg/kg at 67.1% and 150 mg/kg at 64.0%.

Overall, the in vivo PK study of Compound 1 successfully revealed its pharmacokinetic behavior without any observed adverse effects in the animal models.

### Example 4: Drug Doses

The mdx mouse is the most widely used animal model for DMD research. Mdx mice, also referred to as dystrophic mice, do not express dystrophin due to a nonsense point mutation in exon 23. A study was performed to determine efficacious doses of Compound 1 using C57BL/10.ScSn.DMD<mdx> (mdx) mice. Mdx mice were treated for 28 days with vehicle or 1 mg/kg, 3 mg/kg, 10 mg/kg, or 30 mg/kg of Compound 1. All mice received Vehicle on days they were not treated with Compound 1. Mice receiving Compound 1 were treated with the drug for 4 days at the indicated dose, then Vehicle for 3 days (also referred to as 40N/3OFF, or 4/3) and this 4ON/3OFF regimen was repeated for the length of the 28-day experiment. One group of control C57BL/6J (BL/6) mice served as reference.

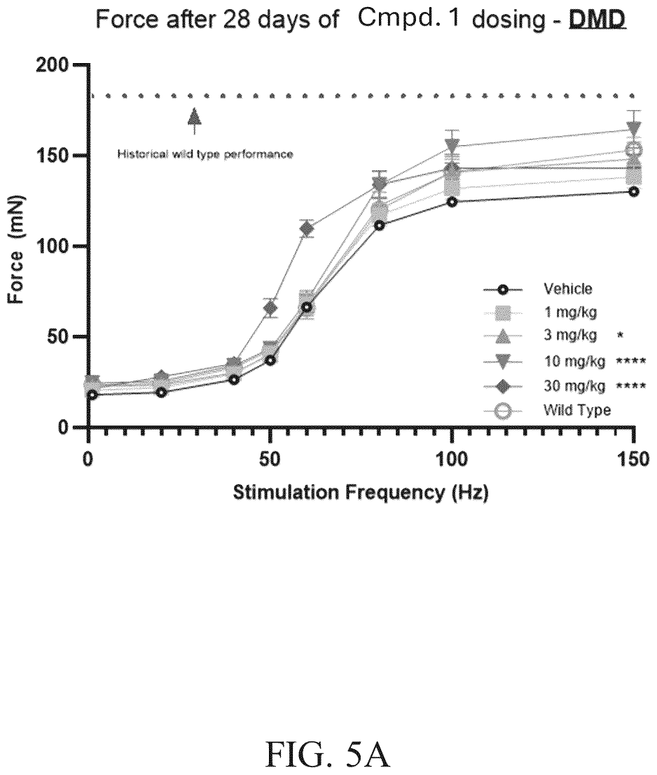

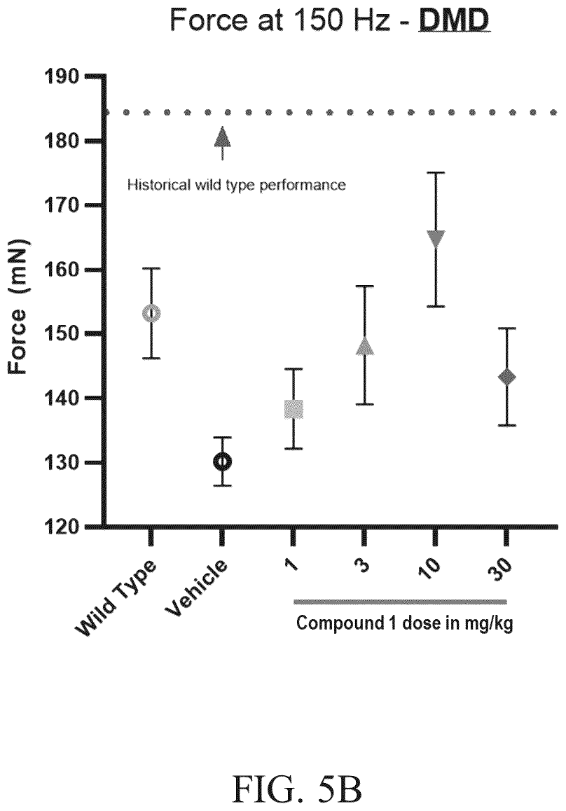

On Day 29, following 4 weeks of treatment respectively, muscle function was measured. As shown in FIGS. 5A and 5B, there was a statistically significant increase in maximal force production with treatment of Compound 1 at all doses except the 1 mg/kg dose, compared to vehicle treated dystrophic mice.

### Example 5: Dosing Regimens in Mdx Mice

A separate study was performed to evaluate muscle function in dystrophic mice administered Compound 1 at different dosing intervals.

### Animals

Table 7 below provides a summary of the different groups in the study, including the number of animals in each group, the compound ID, vehicle, dose level (in mg/kg), dose concentration (in mg/mL), dose volume (in mL/kg), and dose route. Mice were assigned to one of five C57BL/10.ScSn.DMD<mdx> (mdx) groups based on body weight. One group of control C57BL/6J (BL/6) mice served as reference. Mice were treated orally (PO) with either Vehicle or Compound 1 oxalate salt, at different dosing intervals. All mice received Vehicle on days they were not treated. On Days 29 and 43, following 4 and 6 weeks of treatment respectively, muscle function was measured. On Day 57, following 8 weeks of treatment, a final muscle function evaluation was performed, followed by blood collection, euthanasia and tissue collection.

TABLE 7
Dosing Regimen Study Design Details; Cmpd. 1 = Compound 1

| | | | | | Dose | | | | |
|---|---|---|---|---|---|---|---|---|---|
| | | Dose Level | | | Concentration | | | | |
| | | (mg/kg) | | Dose | (mg/mL) | | Dose | | |
| | | | Active | Volume | | Active | Route/ | Number | |
| Group | Treatment | Salt | Moiety | (mL/kg) | Salt | Moiety | Frequency | of Males | Strain |
| 1 | Vehicle | 0 | 0 | 10 | 0 | 0 | P.O. Daily | 10 | mdx/J |
| 2 | Cmpd. 1 | 11.9 | 10 | 10 | 1.19 | 1 | P.O. 4/3 | 10 | mdx/J |
| 3 | Cmpd. 1 | 11.9 | 10 | 10 | 1.19 | 1 | P.O. 5/2 | 10 | mdx/J |
| 4 | Cmpd. 1 | 11.9 | 10 | 10 | 1.19 | 1 | P.O. 6/1 | 10 | mdx/J |
| 5 | Cmpd. 1 | 0.119 | 0.1 | 10 | 0.0119 | 0.01 | P.O. Daily | 10 | mdx/J |
| 6 | Vehicle | 0 | 0 | 10 | 0 | 0 | P.O. Daily | 10 | BL6/J |

Group 1 was dosed with Vehicle daily. Group 2 was dosed with Compound 1 for 4 days, then Vehicle for 3 days (also referred to as 40N/3OFF, or 4/3), repeated throughout the length of the study. Group 3 was dosed with Compound 1 for 5 days, then Vehicle for 2 days (also referred to as 5ON/2OFF, or 5/2). Group 4 was dosed with Compound 1 for 6 days, then Vehicle for 1 days (also referred to as 6ON/1OFF, or 6/1). Group 4 was dosed with Compound 1 daily.

Compound 1 salt dose formulations were prepared by dilution of stock solutions, as appropriate. For the preparation of the Compound 1 oxalate salt stock solution, 357 mg of Compound 1 oxalate salt powder was dissolved into 15 mL of DMSO, forming a 20 mg/mL stock. The stock solution was then individually aliquoted into 250 μL aliquots and stored at −80° C. until use for the Group 2, 3 and 4 dose formulation.

On each day of dosing, individual dose formulations were prepared as follows:

For Groups 1 and 6 (Vehicle), as well as Groups 2-4 non-test article dosing days and for the Group 5 dilution, the dosing vehicle was prepared by diluting 750 μL of DMSO in 14.25 mL of water to create a clear colorless solution (5:95, DMSO:Water).

For Groups 2-4, a single 250 μL aliquot of the 20 mg/mL Compound 1 oxalate salt DMSO Stock Solution was diluted with 4750 μL of Water to create 5 mL of a clear colorless solution at a concentration of 1 mg/mL.

For Group 5, 50 μl of Group 2-4 solution was diluted into 4.95 mL of Vehicle to provide 5 mL of a clear colorless solution at a concentration of 0.01 mg/mL.

Oral formulations were administered by oral gavage using a sterile 20-22 G flexible gavage needle with a soft foam tip. Doses were calculated using pre-treatment body weights (kg) and a dose volume of 10 mL/kg.

### In Vivo Muscle Function

Muscle performance was measured in vivo with a 305C muscle lever system (Aurora Scientific Inc., Aurora, CAN). Once anesthesia in the mouse was accomplished, the mouse was placed on a thermostatically controlled table where anesthesia was maintained via nose-cone (~2-3% isoflurane, or to effect). The right knee was isolated using a pin pressed against the tibial head and the foot firmly fixed to a footplate on the motor shaft. For the plantarflexor muscle group, contractions were elicited by percutaneous electrical stimulation of the sciatic nerve and optimal isometric twitch torque was determined by increasing the current with a minimum of 30 s between each contraction to avoid fatigue. A series of stimulations were then performed at increasing frequency of stimulation (0.2 ms pulse, 500 ms train duration): 1, 20, 40, 50, 60, 80, 100, 150 Hz. Maximal peak isometric force at each frequency, and force-frequency relationship were plotted.

Muscle function data were grouped and means and standard errors calculated. Statistical analysis was performed using GraphPad Prism 10. Data were analyzed using a one-way ANOVA or two-way ANOVA with or without repeated measures as appropriate. Multiple comparisons were performed by the Holm-Sidak post-hoc method with an α=0.05.

### Study Results

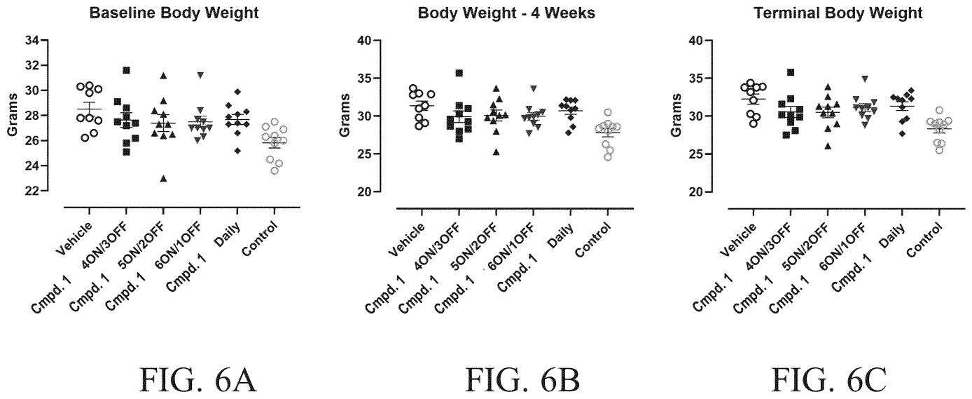

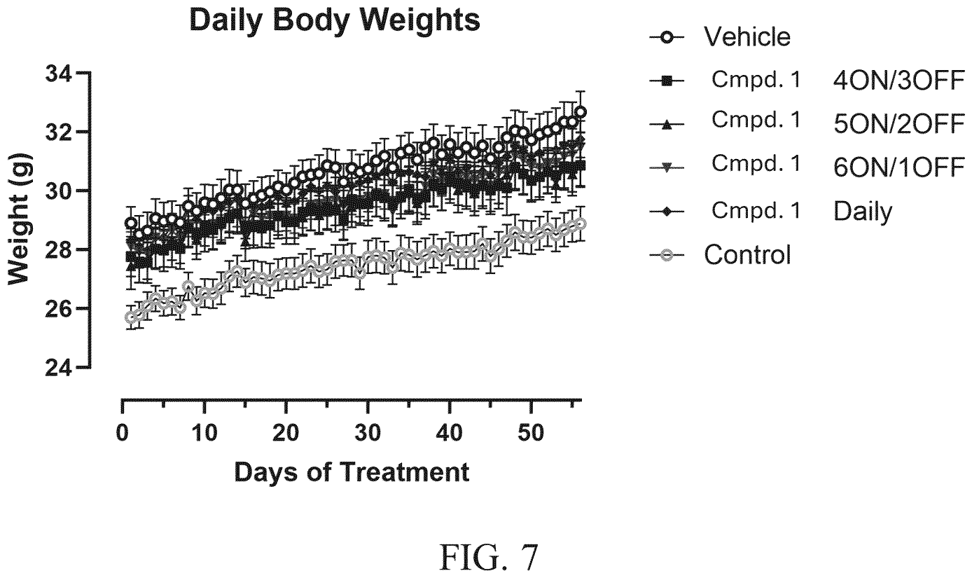

Mice were weighed on dosing days for the duration of the treatment period. As shown in FIG. 6A, dystrophic mice were assigned to groups in such a way that there were no differences in body weights at the start of the study. As shown in FIGS. 6B and 6C, there were no statistically significant differences in average body mass between the experimental groups at the midpoint (after 4 weeks of treatment) and end of the study. Furthermore, as shown in FIG. 7 which shows daily body weights, average body mass trended towards an increase across all groups irrespective of treatment.

### In Vivo Plantarflexor Function

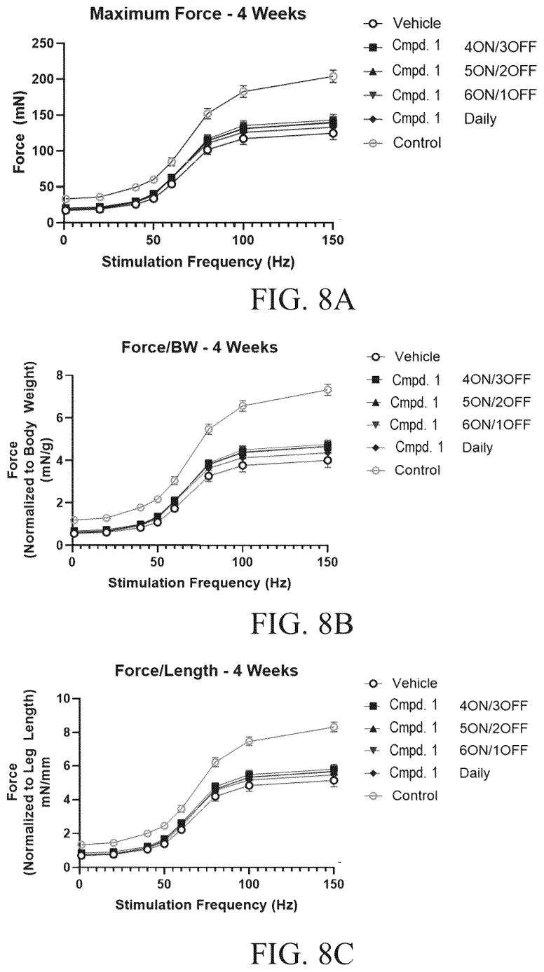

On Day 29, after 4 weeks of treatment, plantarflexion muscle group function was measured. As shown in FIG. 8A, there was a statistically significant increase in maximal force production with treatment of Compound 1 at all dosing intervals except daily, compared to vehicle treated dystrophic mice. Specifically, Vehicle vs. Compound 1 4ON/3OFF had a significance of 0.0044; Vehicle vs. Compound 1 5ON/2OFF had a significance of *0.0129; Vehicle vs. Compound 1 6ON/1OFF had a significance of **0.0017; and Vehicle vs. Compound 1 Daily had no statistical significance (ns) 0.1162. When normalized to body weight, treatment with Compound 1 resulted in a statistically significant increase in maximal force production at all dosing intervals, as shown in FIG. 8B. Specifically, Vehicle vs. Compound 1 4ON/3OFF had a significance of ***0.0001; Vehicle vs. Compound 1 5ON/2OFF had a significance of ***0.0009; Vehicle vs. Compound 1 6ON/1OFF had a significance of ***0.0001; and Vehicle vs. Compound 1 Daily had a significance of *0.0437. When normalized to leg length, treatment with Compound 1 resulted in a statistically significant increase in maximal force production in animals treated on a dosing interval of 4ON/3OFF (Group 2) or 5ON/2OFF (Group 3), as shown in FIG. 8C. Specifically, Vehicle vs. Compound 1 4ON/3OFF had a significance of ***0.0003; Vehicle vs. Compound 1 5ON/2OFF had a significance of *0.0243; Vehicle vs. Compound 1 6ON/1OFF had had no statistical significance (ns) 0.0753; and Vehicle vs. Compound 1 Daily had no statistical significance (ns) 0.0993.

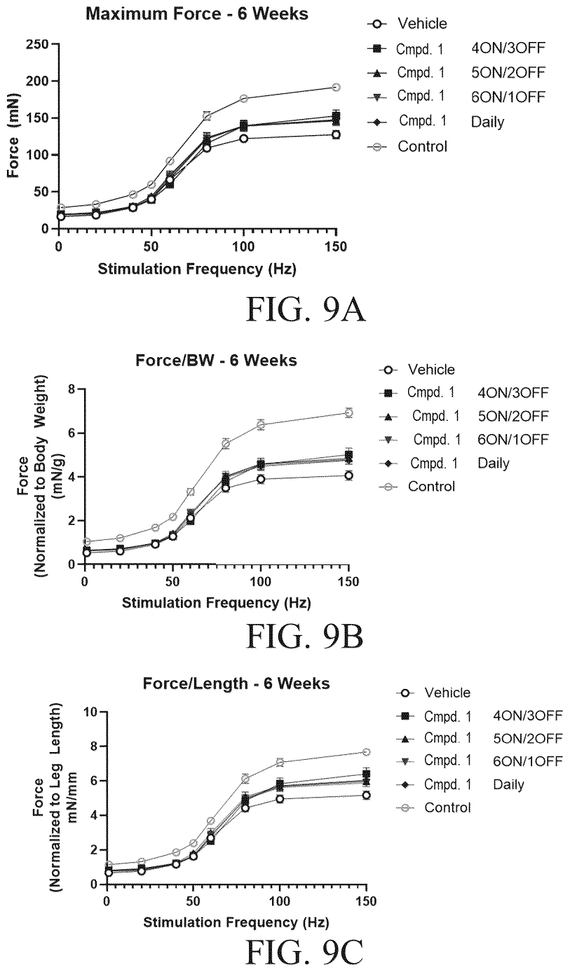

On Day 43, after 6 weeks of treatment, plantarflexion muscle group function was measured again. There was a statistically significant increase in maximal force production with treatment of Compound 1 at all dosing intervals, compared to vehicle treated dystrophic mice, as shown in FIG. 9A. Specifically, Vehicle vs. Compound 1 4ON/3OFF had a significance of *0.0213; Vehicle vs. Compound 1 5ON/2OFF had a significance of **0.0066; Vehicle vs. Compound 1 6ON/1OFF had a significance of **0.0037; and Vehicle vs. Compound 1 Daily had a significance ***0.0005. This increase in maximal force production also persisted with normalization to body weight or leg length, as shown in FIGS. 9B and 9C. For FIG. 9B, specifically, Vehicle vs. Compound 1 4ON/3OFF had a significance of **0.0039; Vehicle vs. Compound 1 5ON/2OFF had a significance of ***0.0004; Vehicle vs. Compound 1 6ON/1OFF had a significance of ***0.0007; and Vehicle vs. Compound 1 Daily had a significance of ***0.0007. For FIG. 9C, specifically, Vehicle vs. Compound 1 4ON/3OFF had a significance of **0.0011; Vehicle vs. Compound 1 5ON/2OFF had a significance of **0.0055; Vehicle vs. Compound 1 6ON/1OFF had a significance of *0.0104; and Vehicle vs. Compound 1 Daily had a significance of ***0.0002.

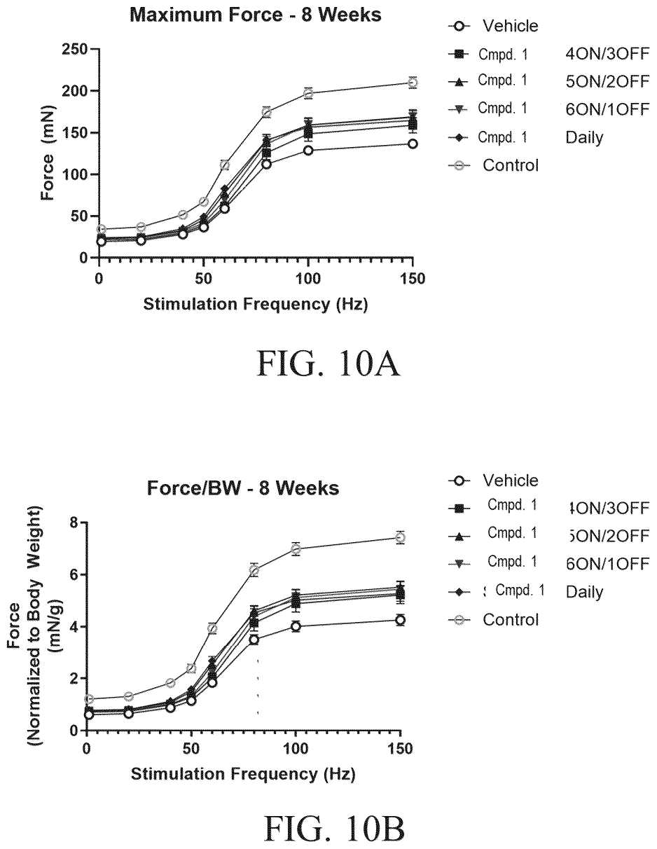

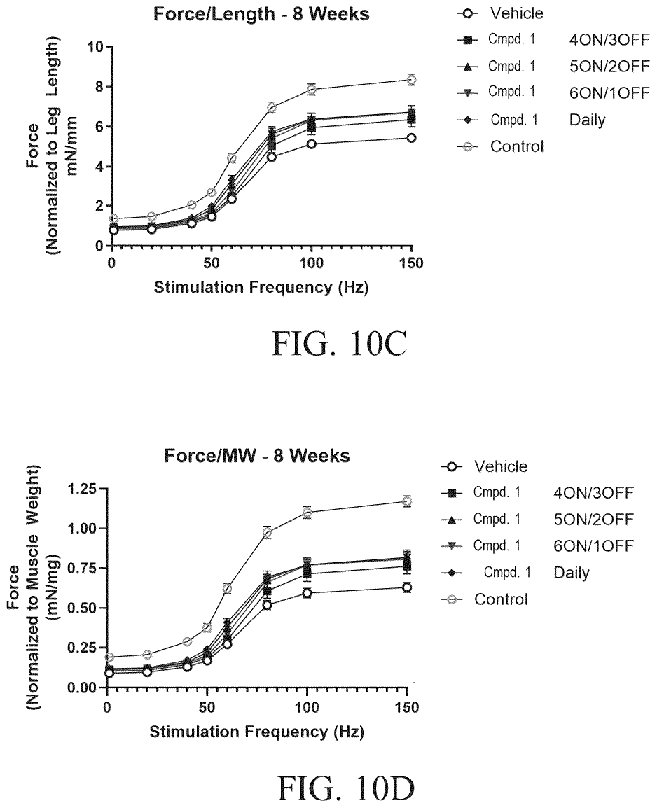

On Day 57, after 8 weeks of treatment (terminal), a final plantarflexion muscle group function assessment was performed. There was a statistically significant increase in maximal force production with treatment of Compound 1 at all dosing intervals compared to vehicle treated dystrophic mice, as shown in FIG. 10A. For FIG. 10A, specifically, Vehicle vs. Compound 1 4ON/3OFF had a significance of *0.0101; Vehicle vs. Compound 1 5ON/2OFF had a significance of ****<0.0001; Vehicle vs. Compound 1 6ON/1OFF had a significance of ****<0.0001; and Vehicle vs. Compound 1 Daily had a significance of <****0.0001. This increase in maximal force production persisted with normalization to body weight, skeletal size, or muscle weight, as shown in FIGS. 10B-D. For FIG. 10B, specifically, Vehicle vs. Compound 1 4ON/3OFF had a significance of ***0.0002; Vehicle vs. Compound 1 5ON/2OFF had a significance of <0.0001; Vehicle vs. Compound 1 6ON/1OFF had a significance of <****0.0001; and Vehicle vs. Compound 1 Daily had a significance of <****0.0001. For FIG. 10C, specifically, Vehicle vs. Compound 1 4ON/3OFF had a significance of **0.0064; Vehicle vs. Compound 1 5ON/2OFF had a significance of <****0.0001; Vehicle vs. Compound 1 6ON/1OFF had a significance of ****<0.0001; and Vehicle vs. Compound 1 Daily had a significance of ****<0.0001. For FIG. 10D, specifically, Vehicle vs. Compound 1 4ON/3OFF had a significance of **0.0012; Vehicle vs. Compound 1 5ON/2OFF had a significance of <*0.0001; Vehicle vs. Compound 1 6ON/1OFF had a significance of ****<0.0001; and Vehicle vs. Compound 1 Daily had a significance of ****<0.0001.

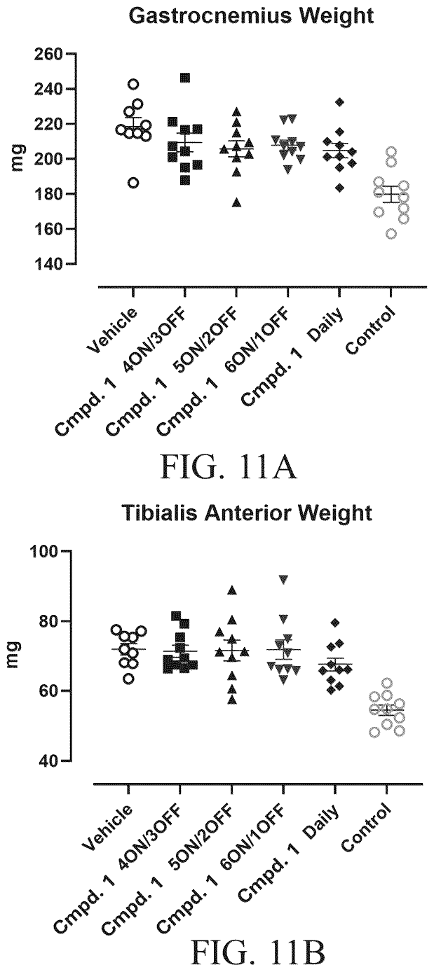

Immediately following muscle function testing and blood collection on Day 57, the mice were euthanized and the gastrocnemius and tibialis anterior muscles were isolated and weighed. The muscles from the contralateral leg were mounted for histology evaluation. All tissues were stored at −80° C. until analysis. There were no differences in gastrocnemius or tibialis anterior muscle weights across groups, as shown in FIGS. 11A and 11B.

In summary, following 4 weeks of treatment, plantarflexor muscle strength was significantly increased for mice dosed with Compound 1 at all dosing intervals except daily when compared to Vehicle controls. This effect was increased at 6 weeks, where muscle strength was significantly increased for mice dosed with Compound 1 at all dosing intervals, and further maintained at 8 weeks. Treatment did not appear to impact muscle size when measured by weight. Collectively, these data show that all dosing interval tested resulted in increase in plantarflexor muscle strength in animals treated with Compound 1 and that dosing intervals where Compound 1 is administered intermittently, such as the 40N/3OFF or 5ON/2OFF intervals, is equally effective as daily administration and may be more effective at earlier treatment timepoints.

### Example 6: Evaluation of Compound 1 in a Phase 1 Clinical Trial

Compound 1, a potent, muscle penetrant, small molecule inhibitor of AAK1, has been shown to significantly increase muscle force in mouse models of Duchenne muscular dystrophy (DMD) and in wild type mice following controlled muscle injury. Based on studies not shown, the maximum tolerated dose (MTD) following 14 days of daily oral gavage was 30 mg/kg/day in the dog and 300 mg/kg/day in the rat.

A First-in-human (FIH), Phase 1 study of orally administered Compound 1 in healthy adult volunteers (HVs) and adult participants with DMD is conducted to determine safety, tolerability, pharmacokinetics and pharmacodynamics.

Part A is a Single Ascending Dose (SAD) part that enrolled HVs in 5 dose cohorts up to 400 mg. Each participant received a single oral dose of Compound 1 or matched placebo on Day 1. Each cohort received a higher dose than the prior cohort.

Part B is a Multiple Ascending Dose (MAD) part that enrolled approximately 32 HVs in 4 sequential dose cohorts. Each participant received a daily oral dose of Compound 1 or matched placebo on Day 1 to Day 7. Each cohort received a higher dose than the prior cohort. Part B participants received one oral dose of Compound 1 daily or matched placebo daily for seven days in accordance with cohort assignment: (1) 60 mg, (2) 120 mg, (3) 180 mg, and (4) 240 mg.

Part C assesses the effect of food on the PK of Compound 1 in a fixed sequence, crossover design. The dose tested is determined by the Safety Review Committee (SRC) following review of safety, tolerability, and available PK and PD data from Part A. Approximately 8 healthy participants who completed Part A at the anticipated dose level (in a fasted state) are croseds over into a subsequent fed cohort and receive a single dose of Compound 1 or matched placebo at the same dose level that they received in Part A, but following a high fat meal.

Part D was an open-label Multiple Dose cohort comprising 5 adult males (ages 20-27) with genetically confirmed DMD. Each participant received 60 mg of Compound 1 once daily for 5 consecutive days every week for a total of 4 weeks (i.e., 20 doses over 28 days).

For the clinical trial, primary and secondary endpoints include, but are not limited to:
(1) incidence and severity of treatment emergent adverse events (TEAEs);
(2) occurrence of clinically significant changes in laboratory assessments, physical examination findings, vital signs, and electrocardiogram (ECG) values;
(3) calculation of plasma PK parameters including but not limited to: maximum concentration (Cₘₐₓ), time to maximum concentration (Tₘₐₓ), area under the curve (AUC) from time 0 to the last measurable concentration (AUC₀₋ₗₐₛₜ), AUC from time 0 to infinity (AUC∞), apparent terminal half-life (t½), apparent clearance (CL/F), apparent terminal volume of distribution (Vz/F) and ratios of Cₘₐₓ and AUC;
(4) change from baseline and placebo-corrected change from baseline in QTcF including exposure response;
(5) change from baseline and placebo corrected change from baseline for other parameters, categorical outliers; and
(6) changes from baseline in upper extremity dynamometry, % predicted forced vital capacity, and 99th percentile maximum effort.

Compound 1 concentrations in plasma with nominal times were evaluated using Phoenix WinNonlin software, version 8.4 (Pharsight Corporation, USA). Noncompartmental analysis was performed (Model type: 200-212) using the extravascular option. All AUC determinations were determined using the ‘linear up-log down trapezoidal’ method.

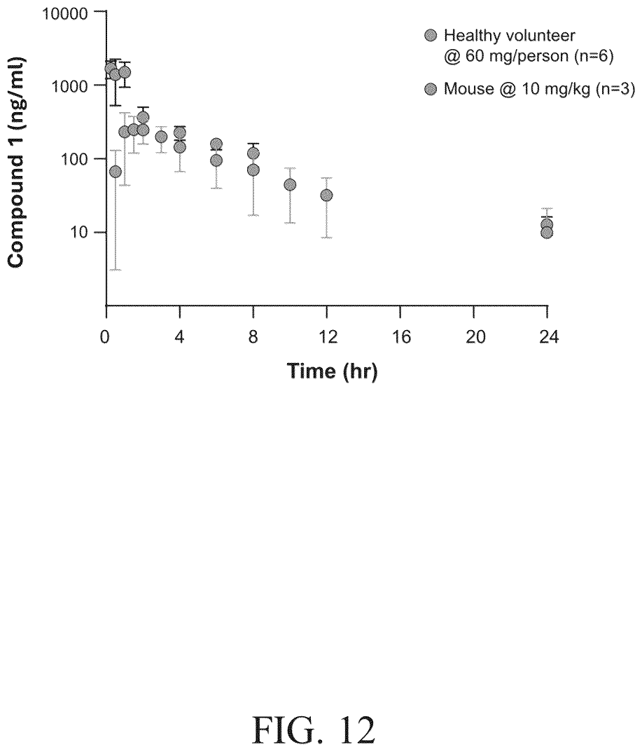

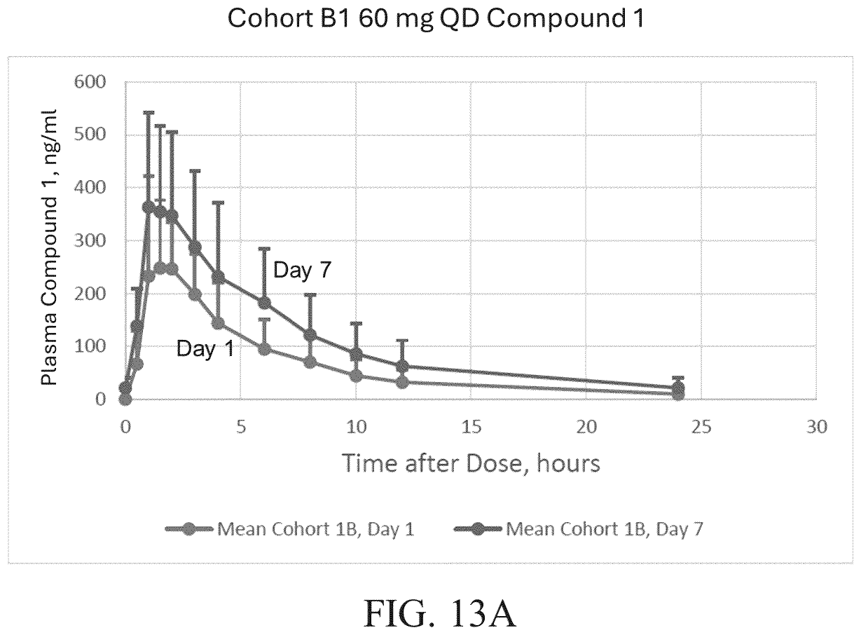

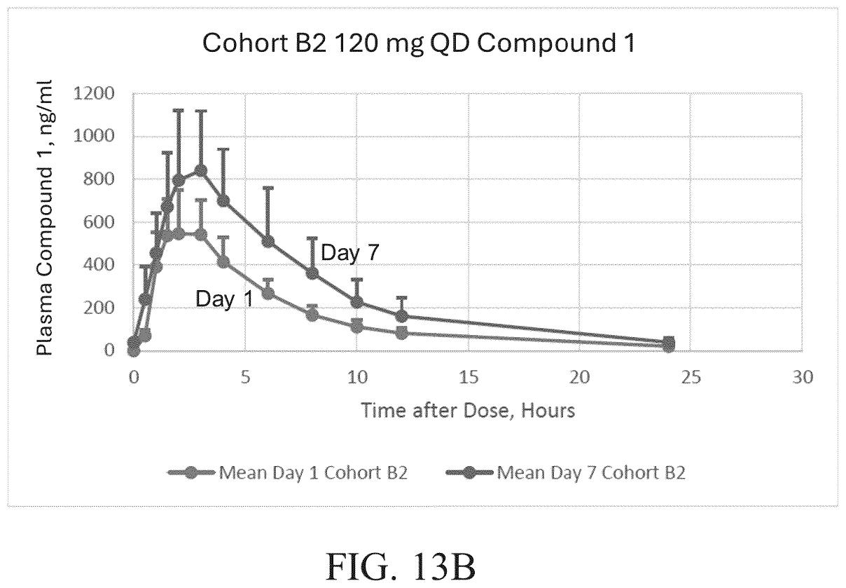

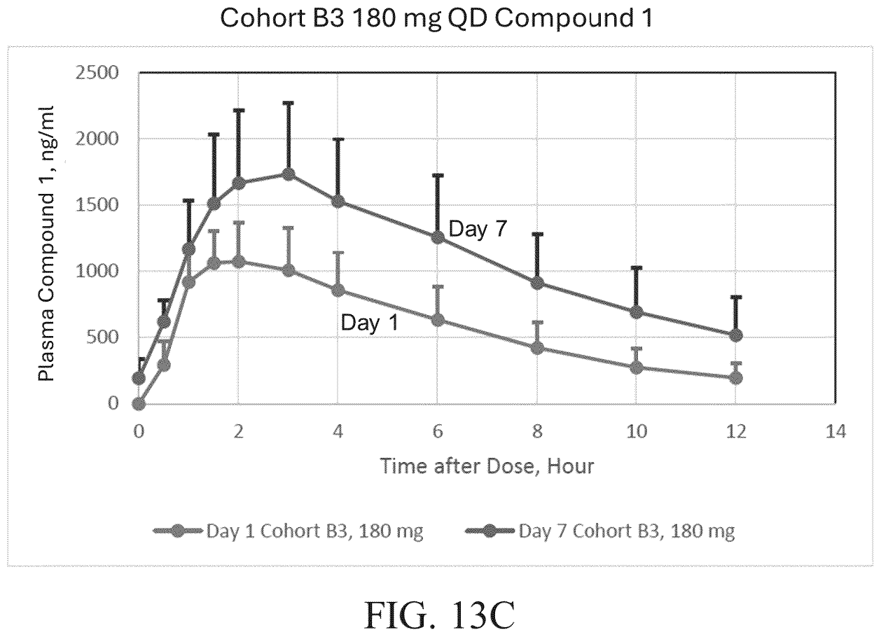

Preliminary results of Part A and Part B show that Compound 1 was well tolerated with no adverse events reported at dosages up to at least 120 mg. In addition, plasma PK levels demonstrated a desired coverage profile following treatment with various dosages in Part B (FIGS. 13A-13C). Compound 1 exposure in healthy volunteer humans treated with 60 mg of Compound 1 was consistent with preclinical efficacious dose in mice (FIG. 12) and demonstrated desired exposure of above the Compound 1 IC₅₀ for 6-8 hours.

### Results of Part D:

Grip strength was measured using the standardized MyoGrip measurement device. Across all study participants, an average doubling of strength from ~2 kg to ~4 kg was observed.
The pharmacokinetic profile of Compound 1 translated to DMD patients.
Compound 1 appeared to be safe and well tolerated in all study participants.
Study participants appeared to remain stable in other exploratory measurement areas.

### Example 7: Synthesis and Characterization of Crystalline Compound 1 Form I Oxalate

Crystalline Compound 1 Form I oxalate was prepared on 100 mg and 1.5 g scale according to the following procedure.

Amorphous Compound 1 (1.5 g) and a magnetic stir bar were charged into a 1-necked flask. To a separate vial, oxalic acid (347 mg) and IPA (20 V) were mixed at room temperature, producing a clear solution. The clear solution was added directly to the amorphous Compound 1 and stirred (500 rpm) at room temperature quickly dissolving the solids, resulting in a clear solution. The mixture was then heated to 70° C. and stirred at that temperature for 1 h. The resulting opaque solution was then cooled to 25° C., producing an immobile slurry. To this was added MTBE (20 V) making the slurry mobile. The suspension was then stirred overnight. The product was isolated by vacuum filtration and washed with an additional portion of MTBE (20 V). The solid was dried under vacuum with nitrogen counterbalance (yield: 1.42 g).

### Example 8: Synthesis and Characterization of Crystalline Compound 1 Form I Hydrochloride

Crystalline Compound 1 Form I hydrochloride was prepared on 50 mg and 300 mg scale according to the following procedure.

300 mg Scale: Amorphous Compound 1 (300 mg) and a magnetic stir bar were charged into a 20 mL vial. To a separate vial, hydrochloric acid (10.3 μL) and IPA (20 V) were mixed at room temperature, producing a clear solution. The clear solution was added directly to the amorphous Compound 1 and stirred (500 rpm) at room temperature. Solids remained undissolved on the sides of the vial. The mixture was then heated to 70° C., fully dissolving the remaining solids, and stirred at that temperature for 1 h. The solution was then cooled to 25° C., yielding a clear solution. To this was added MTBE (20 V) producing an opaque solution that was stirred overnight. The product was isolated by vacuum filtration and then washed with additional MTBE (20 V). The resulting solid was dried under vacuum with nitrogen counterbalance (yield: 263.6 mg).

### Example 9: Synthesis and Characterization of Crystalline Compound 1 Form I Phosphate

Crystalline Compound 1 Form I phosphate was prepared on 100 mg and 1.5 g scale according to the following procedure.

Compound 1 (1.5 g) and a magnetic stir bar were charged into a 1-necked flask. To a separate vial, phosphoric acid (338 μL) and IPA (20 V) were mixed at room temperature, producing a clear solution. The clear solution was added directly to the amorphous Compound 1 and stirred (500 rpm) at room temperature. Solids stick to the sides of the flask. The mixture was then heated to 70° C. As the temperature surpassed 60° C., the solution became clear. The solution was stirred at 70° C. for 1 h and remained clear. The solution was then cooled to 25° C. and remained clear. To this was added MTBE (20 V). The solution remained clear during the addition but became progressively opaquer upon continual stirring overnight. After stirring overnight, the solution became immobile. The product was isolated by vacuum filtration and transferred with an additional portion of MTBE (20 V) that acted as both a wash and to improve the mobility of the suspension for ease of transfer. The solid was dried under vacuum with nitrogen counterbalance (yield: 1.49 g).

### Example 10: Evaluation of Compound 1 in a CTX Injury Model

The effects of Compound 1 on skeletal muscle regeneration were evaluated in MDX mice with CTX-induced muscle injury.

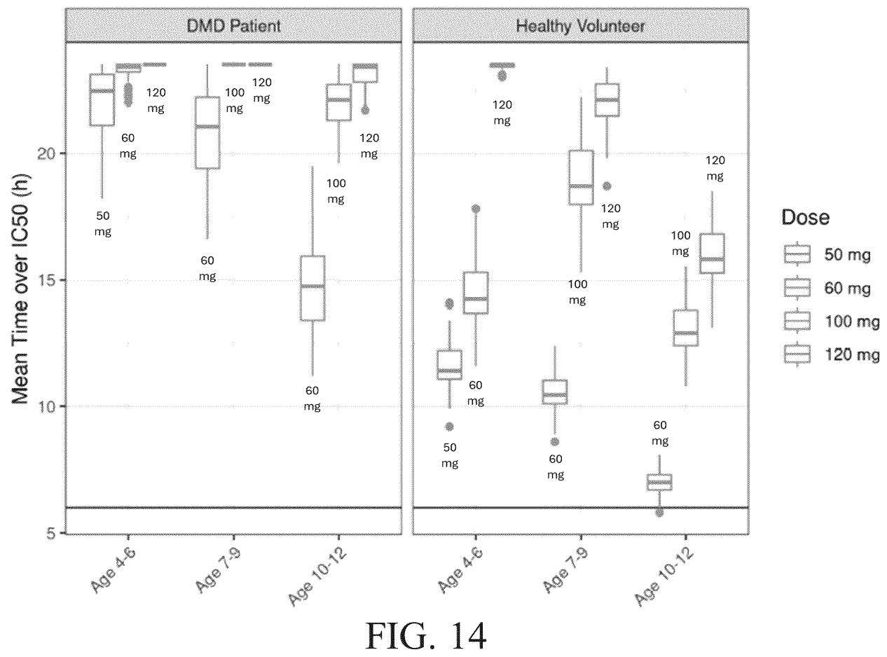

The tibialis anterior (TA) of each MDX mouse was injected with cardiotoxin (CTX) at Day 0 to induce skeletal muscle injury. The mice with CTX-induced muscle injury were then administered Compound 1 intraperitoneally according to the dosing schedule provided in FIG. 14. On Day 2, tissue was collected and cells were sorted for immunostaining. FIGS. 15A and 15B show the staining of polarized and non-polarized satellite cells expressing PARD3 (partition-defective 3) and adaptor protein NUMB. As shown in FIG. 16A, the percentage of polarized satellite cells increased in PARD3 expressing cells of MDX mice treated with Compound 1 in comparison to wild type mice and MDX mice treated with PBS control. Similar observations were noted in NUMB expressing cells of MDX mice treated with Compound 1.

### Example 10: Identifying Dosing for Pediatric Populations

Simulations were performed to help inform dosing of Compound 1 for pediatric populations of various age ranges (age 4-6 years, 7-9 years, 10-12 years) in clinical trials, where Compound 1 is administered for 12 cycles, with each cycle consisting of daily administration of Compound 1 for 5 days, followed by 2 days where Compound 1 is not administered. The goal was to maximize the number of individuals in each age range achieving time above the target IC50 (130 ng/ml) for at least 6 hours, which was the target efficacious PK profile identified in preclinical models. A population PK (popPK) model in healthy volunteers and DMD patients receiving Compound 1 was developed. Simulations using the popPK model were conducted using Simulx 2024R1, including typical parameter estimates with uncertainty. 100 trials of various dosing regimens across various age cohorts (4-6 years, 7-9 years, and 10-12 years) were completed to guide pediatric dose selection. Male individuals were dosed daily for 5 days, with 2 days off. Simulations were performed assuming individuals behaved like healthy volunteers and DMD patients.

The results of the simulations are summarized in Table 8 (DMD patients) and Table 9 (healthy volunteer) and FIG. 14.

TABLE 8
Time Above IC50 for DMD patients

| Cohort | Dose | | Median | Min | Max |
|---|---|---|---|---|---|
| Age 4-6 | 30 | mg | 12.55 | 9.2 | 17.5 |
| Age 4-6 | 50 | mg | 22.45 | 18.2 | 23.5 |
| Age 4-6 | 60 | mg | 23.4 | 22 | 23.5 |
| Age 4-6 | 120 | mg | 23.5 | 23.5 | 23.5 |
| Age 7-9 | 30 | mg | 9.1 | 6.5 | 12.7 |
| Age 7-9 | 60 | mg | 21.05 | 16.6 | 23.5 |
| Age 7-9 | 100 | mg | 23.5 | 23.5 | 23.5 |
| Age 7-9 | 120 | mg | 23.5 | 23.5 | 23.5 |
| Age 10-12 | 30 | mg | 5.9 | 3.6 | 8.3 |
| Age 10-12 | 60 | mg | 14.75 | 11.2 | 19.5 |
| Age 10-12 | 100 | mg | 22.1 | 19.6 | 23.5 |
| Age 10-12 | 120 | mg | 23.4 | 21.7 | 23.5 |

TABLE 9
Time Above IC50 for healthy volunteer

| Cohort | Dose | | Median | Min | Max |
|---|---|---|---|---|---|
| Age 4-6 | 30 | mg | 6 | 4.8 | 7.2 |
| Age 4-6 | 50 | mg | 11.4 | 9.2 | 14.1 |
| Age 4-6 | 60 | mg | 14.25 | 11.6 | 17.8 |
| Age 4-6 | 120 | mg | 23.5 | 23 | 23.5 |
| Age 7-9 | 30 | mg | 3.9 | 2.2 | 4.6 |
| Age 7-9 | 60 | mg | 10.45 | 8.6 | 12.4 |
| Age 7-9 | 100 | mg | 18.7 | 15.3 | 22.2 |
| Age 7-9 | 120 | mg | 22.1 | 18.7 | 23.4 |
| Age 10-12 | 30 | mg | 1.3 | 0.4 | 1.8 |
| Age 10-12 | 60 | mg | 7 | 5.8 | 8.1 |
| Age 10-12 | 100 | mg | 12.9 | 10.8 | 15.5 |
| Age 10-12 | 120 | mg | 15.8 | 13.1 | 18.5 |

Simulations were performed to assess doses required in each age group to achieve an adequate time above IC50 (at least 6 hours and less than 24 hours). Simulations showed that the doses selected (50 mg, 60 mg, 100 mg, and 120 mg) were adequate for all groups. As shown, DMD simulations had greater time above IC50 relative to healthy volunteers, consistent with an increased bioavailability of Compound 1. These simulations informed on optimized doses for pediatric trials, which showed that doses of 50 mg and 60 mg for ages 4-6 years and doses of 60 mg and 100 mg for ages 7-9 and 10-12 years would be predicted to achieve targets and reduce pill burden. Simulations under DMD assumptions showed time over IC50 would be higher than assuming a healthy volunteer population.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

From the foregoing, it will be appreciated that specific embodiments of the invention have been described herein for purposes of illustration, but that various modifications may be made without deviating from the scope of the invention. Accordingly, the invention is not limited except as by the appended claims.

1. A method of treating Duchenne Muscular Dystrophy (DMD) in a subject in need thereof, comprising administering a therapeutically effective amount of Compound 1 or a pharmaceutically acceptable salt or deuterated form thereof for a treatment period, wherein each treatment period comprises two or more consecutive treatment cycles, 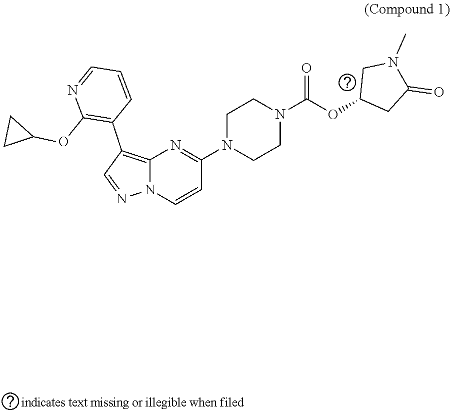 [SMILES: CN1C[C@@H](OC(=O)N2CCN(c3ccn4ncc(-c5cccnc5OC5CC5)c4n3)CC2)CC1=O]
wherein each treatment cycle comprises administering Compound 1 or the pharmaceutically acceptable salt or deuterated form thereof to the subject for 3, 4, 5, 6, 7, 8, 9, or 10 consecutive days, followed by an interval of 1, 2, 3, or 4 days wherein Compound 1 or the pharmaceutically acceptable salt or deuterated form thereof is not administered to the subject.

2. The method of claim 1, wherein:
(a) Compound 1 or the pharmaceutically acceptable salt or deuterated form thereof is administered for at least 3 consecutive days, followed by an interval of at least 1 day wherein Compound 1 or the pharmaceutically acceptable salt thereof is not administered;
(b) Compound 1 or the pharmaceutically acceptable salt or deuterated form thereof is administered for at least 4 consecutive days, followed by an interval of at least 1 day wherein Compound 1 or the pharmaceutically acceptable salt or deuterated form thereof is not administered;
(c) Compound 1 or the pharmaceutically acceptable salt or deuterated form thereof is administered for at least 5 consecutive days, followed by an interval of at least 1 day wherein Compound 1 or the pharmaceutically acceptable salt or deuterated form thereof is not administered; or
(d) Compound 1 or the pharmaceutically acceptable salt or deuterated form thereof is administered for at least 6 consecutive days, followed by an interval of at least 1 day wherein Compound 1 or the pharmaceutically acceptable salt or deuterated form thereof is not administered.

3. The method of claim 1, wherein the treatment period comprises at least one 7-day treatment cycle, wherein Compound 1 or the pharmaceutically acceptable salt or deuterated form thereof is administered for 3 or more days of each 7-day treatment cycle and not administered for 1 or more day of each 7-day treatment cycle.

4. (canceled)

5. The method of claim 3, wherein each treatment cycle is a 7-day treatment cycle wherein:
(a) Compound 1 or the pharmaceutically acceptable salt or deuterated form thereof is administered for 3 consecutive days, followed by an interval of 4 days wherein Compound 1 or the pharmaceutically acceptable salt or deuterated form thereof is not administered;
(b) Compound 1 or the pharmaceutically acceptable salt or deuterated form thereof is administered for 4 consecutive days, followed by an interval of 3 days wherein Compound 1 or the pharmaceutically acceptable salt or deuterated form thereof is not administered;
(c) Compound 1 or the pharmaceutically acceptable salt or deuterated form thereof is administered for 5 consecutive days, followed by an interval of 2 days wherein Compound 1 or the pharmaceutically acceptable salt or deuterated form thereof is not administered; or
(d) Compound 1 or the pharmaceutically acceptable salt or deuterated form thereof is administered for 6 consecutive days, followed by an interval of 1 day wherein Compound 1 or the pharmaceutically acceptable salt or deuterated form thereof is not administered.

6. The method of claim 5, wherein for each 7-day treatment cycle, Compound 1 or the pharmaceutically acceptable salt or deuterated form thereof is administered for 5 consecutive days, followed by an interval of 2 days wherein Compound 1 or the pharmaceutically acceptable salt or deuterated form thereof is not administered.

7. The method of any one of claim 1, wherein Compound 1 or the pharmaceutically acceptable salt or deuterated form thereof is administered once daily.

8. The method of claim 1, wherein 10-400 mg of Compound 1 or the pharmaceutically acceptable salt or deuterated form thereof is administered to the subject daily.

9-10. (canceled)

11. The method of claim 8, wherein about 60 mg of Compound 1 or the pharmaceutically acceptable salt or deuterated form thereof is administered to the subject daily.

12. The method of claim 8, wherein about 120 mg of Compound 1 or the pharmaceutically acceptable salt or deuterated form thereof is administered to the subject daily.

13-17. (canceled)

18. The method of claim 1, wherein Compound 1 or the pharmaceutically acceptable salt or deuterated form thereof is administered orally.

19. The method of claim 1, wherein force generation by muscles is increased, forced vital capacity (FVC) is increased, and/or 99th Percentile Maximum Effort is increased in the subject after administration of Compound 1 or the pharmaceutically acceptable salt or deuterated form thereof.

20-25. (canceled)

26. The method of claim 1, wherein the subject in need thereof is ambulatory.

27-28. (canceled)

29. The method of claim 1, wherein the pharmaceutically acceptable salt of Compound 1 is an oxalate salt.

30. A kit for treatment of Duchenne muscular dystrophy (DMD), comprising at least 3 compound-containing units and optionally at least 1 compound-free unit, wherein each of said compound-containing units comprises Compound 1 or a pharmaceutically acceptable salt thereof, and each of said compound-free unit does not comprise Compound 1 or the pharmaceutically acceptable salt thereof.

31-33. (canceled)

34. The kit of claim 30, wherein each compound-containing unit comprises 10-400 mg of Compound 1 or the pharmaceutically acceptable salt thereof.

35-38. (canceled)

39. A method of increasing the force generation by the skeletal muscles including diaphragm of a subject having Duchenne Muscular Dystrophy (DMD), comprising administering a therapeutically effective amount of Compound 1, or a pharmaceutically acceptable salt thereof, for a treatment period, wherein each treatment period comprises two or more consecutive treatment cycles,
wherein each treatment cycle comprises administering Compound 1 or the pharmaceutically acceptable salt thereof to the subject for 3, 4, 5, 6, 7, 8, 9, or 10 consecutive days, followed by an interval of 1, 2, 3, or 4 days wherein Compound 1 or the pharmaceutically acceptable salt or deuterated form thereof is not administered to the subject.

40. The method of claim 39, wherein each treatment cycle comprises administering Compound 1 or the pharmaceutically acceptable salt or deuterated form thereof to the subject for 5 consecutive days, followed by an interval of 2 days wherein Compound 1 or the pharmaceutically acceptable salt or deuterated form thereof is not administered to the subject.

41. The method of claim 40, wherein about 60 mg or about 120 mg of Compound 1 or the pharmaceutically acceptable salt or deuterated form thereof are orally administered to the subject daily for the 5 consecutive days.

42-44. (canceled)

45. The method of claim 39, wherein the pharmaceutically acceptable salt of Compound 1 is an oxalate salt.

46-48. (canceled)
